SEQUENCE LISTING (3 sequences)

SEQ ID NO: 1 - protein, 199 aa, Homo sapiens
LPICPGGAARCQVTLRDLFDRAVVLSHYIHNLSSEMFSEFDKRYTHGRGFITKAINSCHTSSLATPEDKEQAQQMNQKDFLSLIVSILRSWNEPLYHLVTEVRGMQEAPEAILSKAVEIEEQTKRLLEGMELIVSQVHPETKENEIYPVWSGLPSLQMADEESRLSAYYNLLHCLRRDSHKIDNYLKLLKCRIIHNNNC

SEQ ID NO: 2 - protein, 191 aa, Homo sapiens [sequence omitted]

SEQ ID NO: 3 - protein, 217 aa, Homo sapiens [sequence omitted]

(19) United States
(12) Patent Application Publication
(10) Pub. No.: US 2010/0249029 A1
(43) Pub. Date: Sep. 30, 2010

(54) Peptides with high affinity for the prolactin receptor

(75) Inventors: Jens Breinholt, Dyssegard (DK); Kent Bondensgaard, Vaerlose (DK); Leif Christensen, Roskilde (DK); Leif Nørskov-Lauritzen, Tappernoje (DK); Wang Lingyun, Beijing (CN); Gong Wei, Beijing (CN); Liu Yun, Beijing (CN); Sean Hu, Davis, CA (US); Quinhong Ma, Beijing (CN)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: Novo Nordisk A/S, Bagsvaerd (DK)

(21) Appl. No.: 12/667,778

(22) PCT Filed: Mar. 7, 2008
(86) PCT No.: PCT/EP2008/052784

§ 371 Date: Jun. 8, 2010

Related U.S. Application Data
(60) Provisional application No. 60/958,952, filed on Jul. 10, 2007.
(60) Provisional application No. 61/066,218, filed on Feb. 19, 2008.

(30) Foreign Application Priority Data
Jul. 5, 2007 (EP) 07111799.8
Feb. 13, 2008 (EP) 08101600.8

(51) Int. Cl. A61K 38/16; C07K 14/00; C07H 21/04; C12N 15/74; C12N 1/21; C07K 16/18; A61P 35/00

(52) U.S. Cl. 514/12; 530/350; 536/23.5; 435/320.1; 435/252.33; 530/387.1

(57) ABSTRACT

The invention relates to variants of prolactin, which variants have high affinity for the prolactin receptor.

### FIELD OF THE INVENTION

The present invention relates to variants of prolactin, which variants binds to the prolactin receptor with higher affinity as well as method for producing such variants. Such prolactin variant mutations may be useful for producing prolactin antagonists for use in the treatment of for instance breast cancer.

### BACKGROUND OF THE INVENTION

Prolactin (PRL) is a cytokine with a variety of biological functions, mainly related to lactation, reproduction, osmoregulation and immunoregulation. PRL is a four-helix bundle protein of 199 residues (Somers et al., Nature 372, 478-481 (1994)). The four antiparallel α-helices of the helix bundle are numbered 1-4 as they are defined by the solution structure (PDB code 1 RW5) and occur from the N-terminus of the primary sequence i.e. Helix 1 (residues 15-43), Helix 2 (residues 78-103), Helix 3 (residues 111-137) and Helix 4 (residues 161-193), and PRL furthermore comprises two minor helices denoted Helix 1′ (residues 59-63) and Helix 1″ (residues 69-74), which are present in the loop connecting Helix 1 and Helix 2 (Teilum et al. J. Mol. Biol. 351, 810-823 (2005)), see also FIG. 1.

PRL is a potent growth factor for mammary epithelium and PRL has been associated with the development and growth of breast tumours. Inhibiting pituitary secretion of PRL by dopamine agonists has no effect on breast tumours and it has been established that the tumour is bypassing the effect of the dopamine agonists by utilizing PRL of non-pituitary origin. Thus for treatment of breast cancer it is not sufficient to inhibit the regular pituitary PRL production, whereas a PRL antagonist preventing binding of autocrine PRL to the PRL-R on the tumour, will inhibit the pro-survival and proliferative effect of PRL on the tumour, independently of the source of PRL

PRL binds two molecules of the prolactin receptor (PRL-R) through two regions on PRL referred to as binding site 1 (BS1) and binding site 2 (BS2). The resulting dimerization of the receptor in a 1:2 PRL:PRL-R complex is necessary for activation of the receptor and further signal transduction. A 1:1 complex of PRL:PRL-R, formed through interactions only with the higher affinity BS1 on PRL, is inactive. Thus, variants of PRL solely able to bind via BS1 will have antagonistic properties (see for instance Clevenger et al. Endocr Rev 24, 1 (2003); Goffin et al. Endocr Rev 26, 26 (2005). The soluble, extra cellular domain (ECD) of PRL-R is termed as ECD-PRL-R, and is in the present context (unless specifically noted) referring to Ser-PRLR(1-210).

Even though there is significant homology between PRL and growth hormone, PRL does not bind to the growth hormone receptor (GH-R); however growth hormone (GH) is able to bind both GH-R and PRL-R with different, but overlapping, sites on GH (Cunningham and Wells, Proc. Natl. Acad. Sci. USA 88, 3407 (1991)).

PRL antagonists may be created by interfering with binding of PRL-R to PRL via BS2 for instance by mutating one or more small hydrophobic residues in BS2 to for instance large polar residues (e.g G129R, see for instance Goffin et al. Endocr Rev 26, 26 (2005)) or otherwise interfere with binding of PRL-R to BS2. Such a variant PRL can subsequently only bind PRL-R via BS1 and will thus have attained antagonistic properties. Such variants are also useful for determining the binding of a given peptide to the PRL-R via binding site 1.

Although it has been shown, that the prolactin G129R antagonists can inhibit tumor growth in vivo (Chen et al., Int. J. Oncology 20, 813-818 (2002)), it has also been stated that high level of prolactin receptor antagonists are necessary to obtain effects in vivo (literature (Goffin et al., Endocrine Rev. 26, 400-422 (2005)). Improvement of pharmacokinetic parame-ters could lead to a compound which shows effect in vivo at a dose which is acceptable or desirable for a drug.

In order for an antagonist to compete favourably with wildtype (wt) PRL for BS1, the BS1 binding affinity of the antagonist to BS1 towards PRL-R should be retained, or even improved. Residues within BS1 of the PRL antagonist could for instance be mutated with the purpose of increasing favourable interactions or creating novel interactions in the binding interface with PRL-R at BS1.

BS1 has generally been described to comprise the region bordered by Helix 1 and Helix 4 specifically involving residues Val-23, His-30, Phe-37, Lys-69, Tyr-169, His-173, Arg-176, Arg-177, His-180, Lys-181, Tyr-185, and Lys-187 (Teilum et al. J. Mol. Biol. 351, 810-823 (2005)), These results have been obtained by random mutagenesis of selected PRL residues while screening for mutations that affect PRL-R binding. This is both a lengthy and potentially misleading approach due to, for instance, secondary effects of the mutations. Consequently, the creation of high affinity prolactin receptor antagonists is problematic, since the PRL BS1 has not been precisely identified.

Mutational analysis aimed at identifying residues important for receptor binding has also been performed, see Goffin, V. et al., Mol. Endocrinol. 6, 1381-1392 (1992) and Kinet, S. et al., J. Biol. Chem. 271, 14353-14360 (1996).

### SUMMARY OF THE INVENTION

The present invention is concerned with peptides binding to the prolactin receptor, wherein said peptides have an improved binding via binding site 1 (BS1) to the prolactin receptor.

In one embodiment, the present invention is concerned with an isolated peptide, which peptide is a variant of human prolactin or human growth hormone or human placental lactogen, and which binds to the prolactin receptor, said variant comprising
(i) one or more amino acid mutations in the region corresponding to amino acid residue 24 to 35 of SEQ ID No. 1 and/or
(ia) one or more amino acid mutations in the region corresponding to amino acid residue 52 to 58 of SEQ ID No. 1 and/or
(ib) one or more amino acid mutations in the region corresponding to amino acid residue 50 to 57 of SEQ ID No. 1 and/or
(ii) one or more amino acid mutations in the region corresponding to amino acid residue 66 to 83 of SEQ ID No. 1 and/or
(iii) one or more amino acid mutations in the region corresponding to amino acid residue 176 to 199 of SEQ ID No. 1 and/or
(iv) an addition of from 1 to 5 amino acid residues to the C-terminal.

### DESCRIPTION OF THE DRAWINGS

FIG. 1. The primary sequence (using wt PRL numbering) and secondary structure of vPRL is displayed above the HX analyzed peptides (shown as horizontal bars). Peptides (residues 20-36, 40-63, 66-83, 173-185 and 189-199) identified to comprise BS1 by displaying reduced deuterium incorporation (>0.3 Da) after 1000 s HX in the presence of ECD-PRL-R are colored in grey.

FIG. 2. Deuterium incorporation of vPRL peptides is plotted against time on a logarithmic scale in the presence (triangles) and the absence (circles) of ECD-PRL-R. Apart from peptide 101-113, the peptides shown are a part of BS1 in PRL.

FIG. 3. Sequence alignment of human prolactin, human growth hormone and human placental lactogen. Asterisk (*) denotes identical amino acids, colon (:) denotes structurally and chemically similar amino acids and point (.) denotes amino acids belonging to the same class (in casu hydrophobic or hydrophilic). The sequence listed as “hPRL” is SEQ ID No. 1, the sequence listed as “hGH” is SEQ ID No. 2, and the sequence listed as “hPL” is SEQ ID No. 3.

FIG. 4. Graphical display of results from chemical shift perturbation experiment (data from Table 1). Positive bars represent chemical shift differences observed for amides in free and receptor bound PRL-G129R expressed as ΔCS=[(Δδ_{H})²+(0.1×Δδ_{N})²]^{0.5}. Negative bars represent residues for which backbone amide assignments are missing with shading according to Table 1.

FIG. 5. Biacore assay results of some prolactin analogs. [PRL S61A, Q71A, Q73A, G129R] was a rational designed mutant. [PRL Q73L, M75T, N76S, F80L, G129R] and [PRL S33A, Q73L, G129R, K190R] were two hits identified by SPA assay.

FIG. 6. Ba/F3-PRLR proliferation assay result.

FIG. 7. An example of Ba/F3-PRLR competition assay result.

### DESCRIPTION OF THE INVENTION

The present invention is concerned with peptides binding to the prolactin receptor, wherein said peptides have an improved binding via binding site 1 (BS1) to the prolactin receptor.

In one embodiment, the present invention is concerned with an isolated peptide, which peptide is a variant of a PRL-like cytokine, said variant comprising
(i) one or more amino acid mutations in the region corresponding to amino acid residue 24 to 35 of SEQ ID No. 1 and/or
(ia) one or more amino acid mutations in the region corresponding to amino acid residue 52 to 58 of SEQ ID No. 1 and/or
(ib) one or more amino acid mutations in the region corresponding to amino acid residue 50 to 57 of SEQ ID No. 1 and/or
(ii) one or more amino acid mutations in the region corresponding to amino acid residue 66 to 83 of SEQ ID No. 1 and/or
(iii) one or more amino acid mutations in the region corresponding to amino acid residue 176 to 199 of SEQ ID No. 1 of SEQ ID No. 1 and/or
(iv) an addition of from 1 to 5 amino acid residues to the C-terminal.

For the purpose of this specification, a PRL-like cytokine is a naturally occurring polypeptide ligand which are structurally similar to prolactin having four amphiphatic alpha helices, wherein said natural polypeptide ligand binds to two receptor polypeptides located on the surface of mammalian cells forming a 1:2 complex between the ligand and the receptor polypeptides. Binding of the polypeptide ligand to the receptor polypeptides is through a first polypeptide binding site and a second polypeptide binding site, both binding sites located on the polypeptide ligand. The receptor polypeptides may be same or different. Examples of polypeptide ligands are growth hormone, placental lactogen, interleukin 2, 3, 4, 6, 17, 20, 21, 31, 32 and EPO.

A variant of a given peptide (the parent peptide) is a peptide having an amino acid sequence, which is based on the amino acid sequence of the parent peptide, but carrying one or more amino acid mutations in that sequence, while still retaining at least part of the relevant biological activity of the parent peptide, in this case for instance the ability to bind to the prolactin receptor via binding site 1. Such variant may for instance have substantially the same level of the relevant biological activity as the parent peptide or for instance a significantly higher level of the relevant biological activity. The amino acid mutations in question may be substitutions, additions or deletions or a combination thereof.

In one embodiment, a peptide according to the present invention is capable of binding to the ECD of the prolactin receptor with a KD<10 nM as measured by surface plasmon resonance (SPR), an optical phenomenon that enables detection of unlabeled interactants in real time. The SPR-based biosensors can be used in determination of active concentration, screening and characterization in terms of both affinity and kinetics. In one embodiment, a peptide according to the present invention is capable of binding to the ECD of human prolactin receptor via binding site 1 with a KD<10 nM. In one embodiment, this binding is determined by use of Assay (I) as described herein.

In one embodiment, the PRL-like cytokine comprises an amino acid sequence, which has at least 80% identity to SEQ ID No. 1 including one or more of the amino acid mutations according to the invention. In one embodiment, the PRL-like cytokine has an amino acid sequence having at least 85%, such as at least 90%, for instance at least 95%, such as at least 96%, for instance at least 97%, such as at least 98%, for instance at least 99% identity to SEQ ID No. 1 including one or more of the amino acid mutations according to the invention.

In one embodiment, the PRL-like cytokine comprises an amino acid sequence, which sequence is at least 80% similar to SEQ ID No. 1 including one or more of the amino acid mutations according to the invention. In one embodiment, the PRL-like cytokine has an amino acid sequence, which sequence is at least 85%, such as at least 90%, for instance at least 95%, such as at least 96%, for instance at least 97%, such as at least 98%, for instance at least 99% similar to SEQ ID No. 1 including one or more of the amino acid mutations according to the invention.

In one embodiment, said PRL-like cytokine is human prolactin. The sequence of human prolactin (hPRL) can be seen in SEQ ID No. 1.

In one embodiment, the PRL-like cytokine comprises an amino acid sequence, which has at least 80% identity to SEQ ID No. 2 including one or more of the amino acid mutations according to the invention. In one embodiment, the PRL-like cytokine has an amino acid sequence having at least 85%, such as at least 90%, for instance at least 95%, such as at least 96%, for instance at least 97%, such as at least 98%, for instance at least 99% identity to SEQ ID No. 2 including one or more of the amino acid mutations according to the invention.

In one embodiment, the PRL-like cytokine comprises an amino acid sequence, which sequence is at least 80% similar to SEQ ID No. 2 including one or more of the amino acid mutations according to the invention. In one embodiment, the PRL-like cytokine has an amino acid sequence, which sequence is at least 85%, such as at least 90%, for instance at least 95%, such as at least 96%, for instance at least 97%, such as at least 98%, for instance at least 99% similar to SEQ ID No. 2 including one or more of the amino acid mutations according to the invention.

In one embodiment, said PRL-like cytokine is human growth hormone. The sequence of human growth hormone (hGH) can be seen in SEQ ID No. 2.

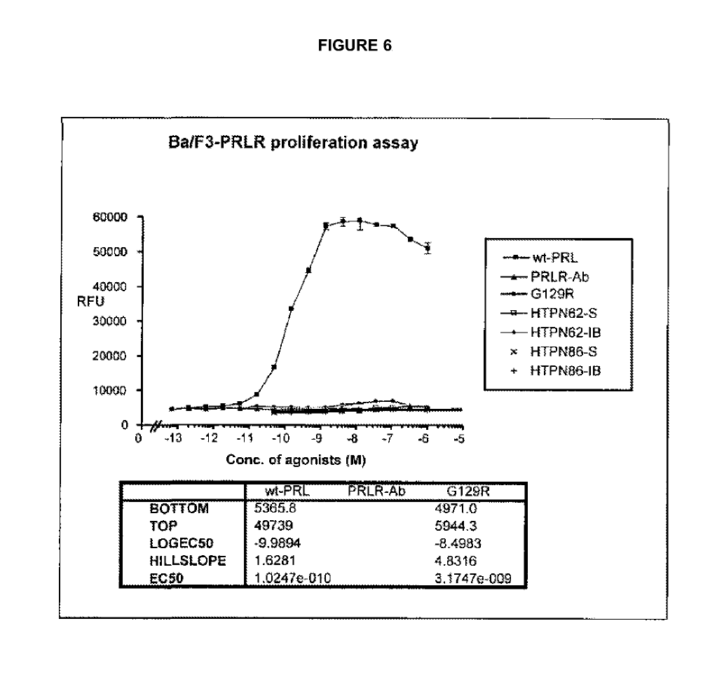

FIG. 6 shows an alignment of growth hormone to prolactin and shows which positions in human growth hormone (hGH, SEQ ID No. 2) corresponds to which positions in human prolactin (hPRL SEQ ID No. 1).

In one embodiment, the PRL-like cytokine comprises an amino acid sequence, which has at least 80% identity to SEQ ID No. 3 including one or more of the amino acid mutations according to the invention. In one embodiment, the PRL-like cytokine has an amino acid sequence having at least 85%, such as at least 90%, for instance at least 95%, such as at least 96%, for instance at least 97%, such as at least 98%, for instance at least 99% identity to SEQ ID No. 3 including one or more of the amino acid mutations according to the invention.

In one embodiment, the PRL-like cytokine comprises an amino acid sequence, which sequence is at least 80% similar to SEQ ID No. 3 including one or more of the amino acid mutations according to the invention. In one embodiment, the PRL-like cytokine has an amino acid sequence, which sequence is at least 85%, such as at least 90%, for instance at least 95%, such as at least 96%, for instance at least 97%, such as at least 98%, for instance at least 99% similar to SEQ ID No. 3 including one or more of the amino acid mutations according to the invention.

In one embodiment, said PRL-like cytokine is human placental lactogen. The sequence of human placental lactogen (hPL) can be seen in SEQ ID No. 3.

FIG. 6 shows an alignment of placental lactogen to prolactin and shows which positions in human placental lactogen (hPL, SEQ ID No. 3) corresponds to which positions in human prolactin (hPRL, SEQ ID No. 1).

The term “peptide” is intended to indicate a sequence of two or more amino acids joined by peptide bonds, wherein said amino acids may be natural or unnatural. The term encompasses the terms polypeptides and proteins, which may consists of two or more polypeptides held together by covalent interactions, such as for instance cysteine bridges, or non-covalent interactions. It is to be understood that the term is also intended to include peptides, which have been derivatized, for instance by the attachment of lipophilic groups, PEG or prosthetic groups. The term peptide includes any suitable peptide and may be used synonymously with the terms polypeptide and protein, unless otherwise stated or contradicted by context; provided that the reader recognize that each type of respective amino acid polymer-containing molecule may be associated with significant differences and thereby form individual embodiments of the present invention (for example, a peptide such as an antibody, which is composed of multiple polypeptide chains, is significantly different from, for example, a single chain antibody, a peptide immunoadhesin, or single chain immunogenic peptide). Therefore, the term peptide herein should generally be understood as referring to any suitable peptide of any suitable size and composition (with respect to the number of amino acids and number of associated chains in a protein molecule). Moreover, peptides in the context of the inventive methods and compositions described herein may comprise non-naturally occurring and/or non-L amino acid residues, unless otherwise stated or contradicted by context.

The term peptide, unless otherwise stated or contradicted by context, (and if discussed as individual embodiments of the term(s) polypeptide and/or protein) also encompasses derivatized peptide molecules. Briefly, in the context of the present invention, a derivative is a peptide in which one or more of the amino acid residues of the peptide have been chemically modified (for instance by alkylation, acylation, ester formation, or amide formation) or associated with one or more non-amino acid organic and/or inorganic atomic or molecular substituents (for instance a polyethylene glycol (PEG) group, a lipophilic substituent (which optionally may be linked to the amino acid sequence of the peptide by a spacer residue or group such as β-alanine, γ-aminobutyric acid (GABA), L/D-glutamic acid, succinic acid, and the like), a fluorophore, biotin, a radionuclide, etc.) and may also or alternatively comprise non-essential, non-naturally occurring, and/or non-L amino acid residues, unless otherwise stated or contradicted by context (however, it should again be recognized that such derivatives may, in and of themselves, be considered independent features of the present invention and inclusion of such molecules within the meaning of peptide is done for the sake of convenience in describing the present invention rather than to imply any sort of equivalence between naked peptides and such derivatives). Non-limiting examples of such amino acid residues include for instance 2-aminoadipic acid, 3-amino-adipic acid, β-alanine, β-aminopropionic acid, 2-aminobutyric acid, 4-aminobutyric acid, 6-aminocaproic acid, 2-aminoheptanoic acid, 2-aminoisobutyric acid, 3-aminoisobutyric acid, 2-aminopimelic acid, 2,4-diaminobutyric acid, desmosine, 2,2′-diaminopimelic acid, 2,3-di-aminopropionic acid, N-ethylglycine, N-ethylasparagine, hydroxylysine, allohydroxylysine, 3-hydroxyproline, 4-hydroxyproline, isodesmosine, alloisoleucine, N-methylglycine, N-methyl-isoleucine, 6-N-methyllysine, N-methylvaline, norvaline, norleucine, ornithine, and statine halogenated amino acids.

The term “identity” as known in the art, refers to a relationship between the sequences of two or more peptides, as determined by comparing the sequences. In the art, “identity” also means the degree of sequence relatedness between peptides, as determined by the number of matches between strings of two or more amino acid residues. “Identity” measures the percent of identical matches between the smaller of two or more sequences with gap alignments (if any) addressed by a particular mathematical model or computer program (i.e., “algorithms”). Identity of related peptides can be readily calculated by known methods. Such methods include, but are not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; Sequence Analysis Primer, Gribskov, M. and Devereux, J., eds., M. Stockton Press, New York, 1991; and Carillo et al., SIAM J. Applied Math. 48, 1073 (1988).

Preferred methods to determine identity are designed to give the largest match between the sequences tested. Methods to determine identity are described in publicly available computer programs. Preferred computer program methods to determine identity between two sequences include the GCG program package, including GAP (Devereux et al., Nucl. Acid. Res. 12, 387 (1984); Genetics Computer Group, University of Wisconsin, Madison, Wis.), BLASTP, BLASTN, and FASTA (Altschul et al., J. Mol. Biol. 215, 403-410 (1990)). The BLASTX program is publicly available from the National Center for Biotechnology Information (NCBI) and other sources (BLAST Manual, Altschul et al. NCB/NLM/NIH Bethesda, Md. 20894; Altschul et al., supra). The well known Smith Waterman algorithm may also be used to determine identity.

For example, using the computer algorithm GAP (Genetics Computer Group, University of Wisconsin, Madison, Wis.), two peptides for which the percent sequence identity is to be determined are aligned for optimal matching of their respective amino acids (the “matched span”, as determined by the algorithm). A gap opening penalty (which is calculated as 3.times. the average diagonal; the “average diagonal” is the average of the diagonal of the comparison matrix being used; the “diagonal” is the score or number assigned to each perfect amino acid match by the particular comparison matrix) and a gap extension penalty (which is usually 1/10 times the gap opening penalty), as well as a comparison matrix such as PAM 250 or BLOSUM 62 are used in conjunction with the algorithm. A standard comparison matrix (see Dayhoff et al., Atlas of Protein Sequence and Structure, vol. 5, supp. 3 (1978) for the PAM 250 comparison matrix; Henikoff et al., Proc. Natl. Acad. Sci. USA 89, 10915-10919 (1992) for the BLOSUM 62 comparison matrix) is also used by the algorithm.

Preferred parameters for a peptide sequence comparison include the following:

Algorithm: Needleman et al., J. Mol. Biol. 48, 443-453 (1970); Comparison matrix: BLOSUM 62 from Henikoff et al., PNAS USA 89, 10915-10919 (1992); Gap Penalty: 12, Gap Length Penalty: 4, Threshold of Similarity: 0.

The GAP program is useful with the above parameters. The aforementioned parameters are the default parameters for peptide comparisons (along with no penalty for end gaps) using the GAP algorithm.

The term “similarity” is a concept related to identity, but in contrast to “identity”, refers to a sequence relationship that includes both identical matches and conservative substitution matches. If two polypeptide sequences have, for example, (fraction ( 10/20)) identical amino acids, and the remainder are all non-conservative substitutions, then the percent identity and similarity would both be 50%. If, in the same example, there are 5 more positions where there are conservative substitutions, then the percent identity remains 50%, but the percent similarity would be 75% ((fraction ( 15/20))). Therefore, in cases where there are conservative substitutions, the degree of similarity between two polypeptides will be higher than the percent identity between those two polypeptides.

Conservative modifications a peptide comprising an amino acid sequence of SEQ ID No. 1 (or SEQ ID No. 2) (and the corresponding modifications to the encoding nucleic acids) will produce peptides having functional and chemical characteristics similar to those of a peptide comprising an amino acid sequence of SEQ ID No. 1 (or SEQ ID No. 2). In contrast, substantial modifications in the functional and/or chemical characteristics of peptides according to the invention as compared to a peptide comprising an amino acid sequence of SEQ ID No. 1 (or SEQ ID No. 2) may be accomplished by selecting substitutions in the amino acid sequence that differ significantly in their effect on maintaining (a) the structure of the molecular backbone in the area of the substitution, for example, as a sheet or helical conformation, (b) the charge or hydrophobicity of the molecule at the target site, or (c) the bulk of the side chain.

For example, a “conservative amino acid substitution” may involve a substitution of a native amino acid residue with a normative residue such that there is little or no effect on the polarity or charge of the amino acid residue at that position. Furthermore, any native residue in the polypeptide may also be substituted with alanine, as has been previously described for “alanine scanning mutagenesis” (see, for example, MacLennan et al., Acta Physiol. Scand. Suppl. 643, 55-67 (1998); Sasaki et al., Adv. Biophys. 35, 1-24 (1998), which discuss alanine scanning mutagenesis).

Desired amino acid substitutions (whether conservative or non-conservative) may be determined by those skilled in the art at the time such substitutions are desired. For example, amino acid substitutions can be used to identify important residues of the peptides according to the invention, or to increase or decrease the affinity of the peptides described herein for the receptor in addition to the already described mutations.

Naturally occurring residues may be divided into classes based on common side chain properties:

1) hydrophobic: norleucine, Met, Ala, Val, Leu, Iie;

2) neutral hydrophilic: Cys, Ser, Thr, Asn, Gln;

3) acidic: Asp, Glu;

4) basic: His, Lys, Arg;

5) residues that influence chain orientation: Gly, Pro; and

6) aromatic: Trp, Tyr, Phe.

In making such changes, the hydropathic index of amino acids may be considered. Each amino acid has been assigned a hydropathic index on the basis of their hydrophobicity and charge characteristics, these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is understood in the art. Kyte et al., J. Mol. Biol., 157, 105-131 (1982). It is known that certain amino acids may be substituted for other amino acids having a similar hydropathic index or score and still retain a similar biological activity. In making changes based upon the hydropathic index, the substitution of amino acids whose hydropathic indices are within .±2 is preferred, those that are within ±1 are particularly preferred, and those within ±0.5 are even more particularly preferred. The greatest local average hydrophilicity of a protein, as governed by the hydrophilicity of its adjacent amino acids, correlates with its immunogenicity and antigenicity, i.e., with a biological property of the protein.

The following hydrophilicity values have been assigned to amino acid residues: arginine (+3.0); lysine (′3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). In making changes based upon similar hydrophilicity values, the substitution of amino acids whose hydrophilicity values are within ±2 is preferred, those that are within ±1 are particularly preferred, and those within ±0.5 are even more particularly preferred.

Peptides of the present invention may also include non-naturally occurring amino acids.

The use of Hydrogen Exchange Mass Spectrometry (HX-MS) and Nuclear Magnetic Resonance (NMR) technology has now enabled a structural definition of the PRL BS1, which provides an excellent tool for creation of high affinity prolactin antagonists. Therefore, the amino acid residues in this region are target for mutagenisis with the purpose of increasing favourable interactions or creating novel interactions in the binding interface with PRL-R at BS1.

A peptide according to the invention may furthermore comprise one or more amino acid mutations, which stabilizes the structure of the prolactin molecule. Such mutations may for instance be mutations, which stabilizes the secondary structure of the prolactin molecule (the stabilization may be determined by use of HX-MS technology). One or more of said amino acid mutation(s) may for instance stabilize the 4-helix bundle structure in prolactin or improve the helix capping in helix 1, helix 2, helix 3 and/or helix 4 of PRL. Such amino acid mutation(s) may also introduce salt bridges in helical segments exposed to solvent. Two or more of said amino acid mutation(s) may also introduce non-native disulfide bonds into prolactin. Such amino acid mutation(s) may also be a substitution of a solvent exposed hydrophobic residue with a polar residue or for instance improve the packing interactions at the hydrophobic core of the 4-helix bundle structure.

The HX-MS technology exploits that hydrogen exchange (HX) of a protein can readily be followed by mass spectrometry (MS). By replacing the aqueous solvent containing hydrogen with aqueous solvent containing deuterium, incorporation of a deuterium atom at a given site in a protein will give rise to an increase in mass of 1 Da. This mass increase can be monitored as a function of time by mass spectrometry in quenched samples of the exchange reaction.

One use of HX-MS is to probe for sites involved in molecular interactions by identifying regions of reduced hydrogen exchange upon protein-protein complex formation. Usually, binding interfaces will be revealed by marked reductions in hydrogen exchange due to steric exclusion of solvent.

Protein-protein complex formation may be detected by HX-MS simply by measuring the total amount of deuterium incorporated in either protein members in the presence and absence of the respective binding partner as a function of time. Furthermore, the deuterium labels can be sub-localized to specific regions of either protein by proteolytic fragmentation of the deuterated protein sample into short peptides and analysis of the deuteron content of each peptide. Peptides that display altered deuterium levels in the presence of binding partner either constitute or are structurally linked to the binding interface (for a recent review on the HX-MS technology see Wales and Engen, Mass Spectrom. Rev. 25, 158 (2006)). A relevant example of application of the HX-MS technology may be found in Horn et al., Biochemistry 45, 8488-8498 (2006).

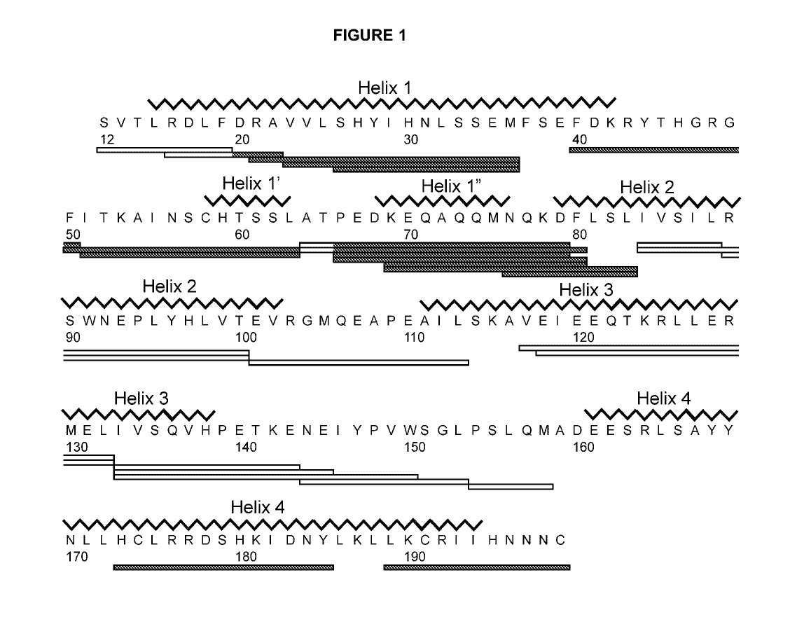

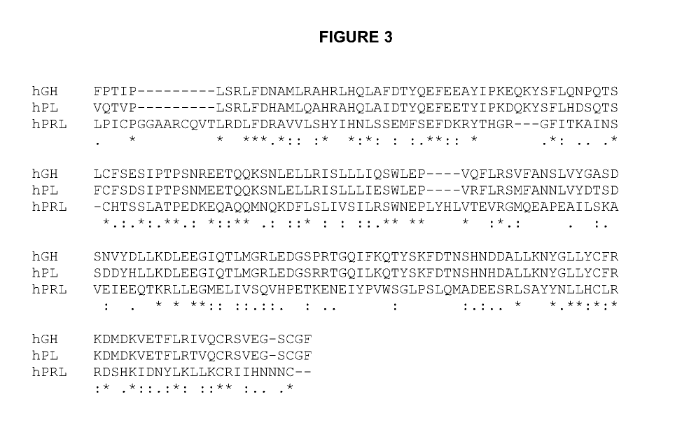

The HX-MS technology used provides information about which surface exposed amide hydrogens in PRL or variants thereof that become shielded from exchange with solvent upon PRL-R binding thereby facilitating a mapping of the binding interface. In addition to this information, however, the methodology can also reveal more indirect structural effects in PRL or variants thereof that give rise to altered HX upon binding. Examples of raw data and the resulting HX-time course plots of peptides from a variant of PRL (vPRL) are shown in FIG. 1 and FIG. 3.

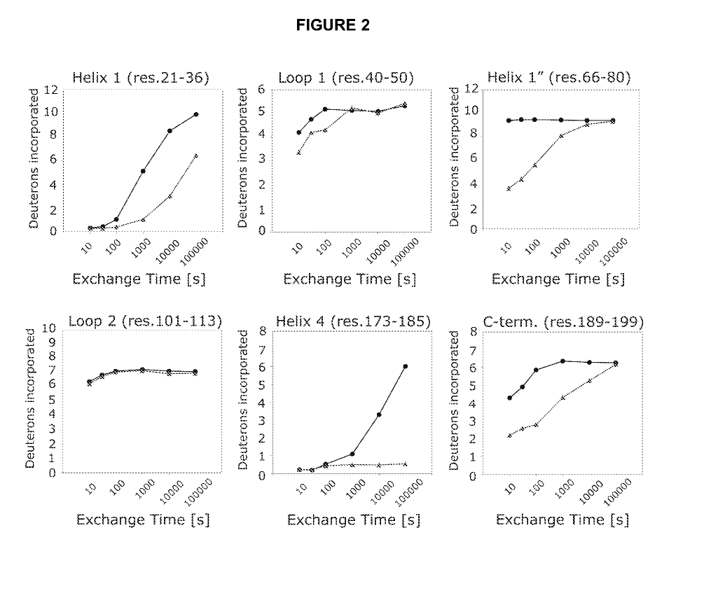

It was surprisingly found that BS1 is larger than previously known and that BS1 includes residues from helix 1″ and the second half of the loop between Helix 1 and Helix 2 (residues 66-83) and the S—S bonded C-terminus (residues 189-199). For the purpose of this specification, BS1 is said to comprise residues within the segments of PRL consisting of amino acid residues 21-36, 40-63, 66-83, 173-199 (FIG. 1 and FIG. 2).

The residues in these regions are readily substituted/modified to increase binding affinity of PRL-R to BS1. In general, candidates for modifications may be substituted by residues of the same group of amino acid residues as the native residue or a closely related group so as not to cause large perturbations of the structural integrity of the respective segments of BS1.

The decreased HX rates observed for amides hydrogens in Helix 2 and Helix 3 constitutes an example of indirect effects propagated through PRL structure upon binding of PRL-R. A reduction in HX rates of Helix 2 and Helix 3, which are located on the opposite side of the PRL molecule and distal to BS1, shows that these regions are stabilized indirectlyduring binding of PRL-R at BS1. The effects observed in Helix 2 and Helix 3 indicate that PRL is significantly stabilized by PRL-R binding at BS1.

Therefore, having detected indirect receptor induced structural effects that stabilize the four-helix bundle structure of PRL, it is observed that one could inherently stabilize any of the helices of PRL by mutagenesis and thereby favour the bound form of PRL. This would increase binding affinity of PRL to PRL-R preferentially through BS1. This could, as mentioned above, for instance be combined with destructive mutations in the BS2 binding interface.

Nuclear Magnetic Resonance (NMR) spectroscopy is a well established technique for characterizing binding interfaces of protein-protein complexes in solution. Most methods are based on ¹H, ¹⁵N-correlation spectroscopy and include chemical shift perturbation (Foster, M. P. et al., Journal of Biomolecular NMR 12, 51-71 (1998)), hydrogen-deuterium exchange (H-D) (Paterson, Y. et al., Science 249, 755-759 (1990)), and cross-saturation (Takahashi, H. et al., Nature Structural Biology 7, 220-223 (2000)) measurements of back bone amide groups. Since both the magnetic environment (chemical shift) and exchange rate constants of amide protons can be affected by direct molecular contacts as well as conformational rearrangements and changed dynamic properties, the chemical shifts perturbation and H-D exchange methods do not distinguish between primary effects originating from direct molecular contacts at the protein-protein interface, and indirect effects attributable to changes in structure and/or dynamics induced by complex formation. In contrast, the cross saturation method relies on transfer of magnetization from one molecule to the other via short-range proton-proton contacts, and secondary effects do not interfere. The cross saturation method then (ideally) uniquely identifies back bone amide groups situated within a distance shorter than 5-7 Å of the interface. Examples of chemical shift perturbation and cross-saturation experiments applied in the characterisation of BS1 are detailed in Example 2.

The overall site 1 binding interface determined by the NMR methods is generally in accordance with results from mutation experiments aimed at identifying residues important for receptor binding (Goffin, V. et al., Mol. Endocrinol. 6, 1381-1392 (1992) and Kinet, S. et al., J. Biol. Chem. 271, 14353-14360 (1996)).

However, as with HX-MS, NMR data indicates additional important receptor interactions. Surprisingly, a strong cross-saturation effect is observed for the C-terminal Cystine (C199), which most likely makes direct contact with the receptor molecule. The proposed receptor interaction involving the C-terminal fragment is further supported by the structural stabilization and reduced amide proton exchange rate observed for the H195-N198 segment as described in Example 2. Furthermore, strong cross-saturation effects are observed for 155 and N56 situated in the loop between helix 1 and helix 2, again in accordance with the amide protons in the 151-S57 region being stabilized and shielded from solvent exchange upon receptor complex formation.

Mutations that would increase stabilization of the four-helix bundle structure in PRL include stabilization of the terminal part of any of the four helices of PRL (so-called helix capping) (including mutations such as E162D, A111D, A111N, A111S, and A111T), introducing new saltbridges in solvent exposed helical segments of PRL (including mutations such as N92D, E162D, A111D, A111N, A111S, and A111T), introduction of new S—S disulfide bonds (including mutations such as L81C/V134C, L88C/L127C, V102/L113C, L95C/E120C, S90C/Y147C, L32C/I119C, D160C/S193C, L81C/V134C, M105C/A108C, M36C/K115C, S33C/L175C, A22C/G129C, H59C/P148C, F37C/L172C, S26C/D183C, S33C/S179C P66C/Q71C or P66C/A72C, V23C/L186C, V99C/A116C, V102C/L113C, S57C/N170C, R89C/Y147C, S82C/E143C, H195C/N198C, K190C/N198C, S33C/R176C, H138C/T141C, M158C/R164C, E93C/W150C, S86C/I146C, V85C/N144C, S82C/N144C, K78C/K142C, K78C/H138C, Q77C/V137C, L63C/S86C, T45C/151C, L1C/S135C). In conjunction, one might also increase the stability of PRL by replacing solvent exposed hydrophobic residues by polar residues (including mutations such as I146S and V149S). Similarly, one might also increase the stability of PRL by improving the packing interactions at the hydrophobic core of the 4-helix bundle structure (including mutations such as L95V/1119V/L175P).

In one embodiment, a peptide according to the present invention carries a substitution mutation in one or more of the positions corresponding to amino acid residues 25, 28, 31, 33, 51, 52, 55, 56, 57, 68, 70, 73, 75, 76, 80, 182, 190, 194, 195, 196 and 197 of SEQ ID No. 1, wherein any substitution in the position corresponding to amino acid residue 73 is not a substitution with alanine.

In one embodiment, a peptide according to the present invention carries a substitution mutation in one or more of the positions corresponding to amino acid residues 25, 28, 31, 33, 51, 52, 55, 56, 57, 68, 70, 73, 75, 76, 80, 182, 190, 194, 195, 196 and 197 of SEQ ID No. 1, wherein the substitution in the position corresponding to amino acid residue 73 is a substitution with a leucine.

In one embodiment, a peptide according to the present invention carries a substitution mutation in one or more of the positions corresponding to amino acid residues 25, 28, 31, 33, 51, 52, 55, 56, 57, 68, 70, 75, 76, 80, 182, 190, 194, 195, 196 and 197 of SEQ ID No. 1.

In one embodiment, the amino acid residue in the position corresponding to amino acid residue 25 of SEQ ID No. 1 has been substituted with a Gln.

In one embodiment, the amino acid residue in the position corresponding to amino acid residue 28 of SEQ ID No. 1 has been substituted with an Asn.

In one embodiment, a peptide according to the present invention carries a substitution mutation in one or more of the positions corresponding to amino acid residues 31, 33, 68, 70, 75, 76, 80, 182, 190, 194, 195, 196 and 197 of SEQ ID No. 1.

In one embodiment, the amino acid residue in the position corresponding to amino acid residue 70 of SEQ ID No. 1 has been substituted with a Lys.

In one embodiment, a peptide according to the present invention carries a substitution mutation in one or more of the positions corresponding to amino acid residues 31, 33, 68, 75, 76, 80, 182, 190, 194, 195, 196, and 197 of SEQ ID No. 1.

In one embodiment, a peptide according to the present invention carries a substitution mutation in one or more of the positions corresponding to amino acid residues 31, 33, 68, 75, 76, 80, 182, 190, 194, 195, 196, and 197 of SEQ ID No. 1.

In one embodiment, the amino acid residue in the position corresponding to amino acid residue 33 of SEQ ID No. 1 has been substituted with an Ala.

In one embodiment, the amino acid residue in the position corresponding to amino acid residue 33 of SEQ ID No. 1 has been substituted with an Asp.

In one embodiment, the amino acid residue in the position corresponding to amino acid residue 75 of SEQ ID No. 1 has been substituted with a Thr.

In one embodiment, the amino acid residue in the position corresponding to amino acid residue 76 of SEQ ID No. 1 has been substituted with a Ser.

In one embodiment, the amino acid residue in the position corresponding to amino acid residue 80 of SEQ ID No. 1 has been substituted with a Leu.

In one embodiment, the amino acid residue in the position corresponding to amino acid residue 182 of SEQ ID No. 1 has been substituted with a Val.

In one embodiment, the amino acid residue in the position corresponding to amino acid residue 194 of SEQ ID No. 1 has been substituted with a Val.

In one embodiment, the amino acid residue in the position corresponding to amino acid residue 195 of SEQ ID No. 1 has been substituted with a Tyr.

In one embodiment, the amino acid residue in the position corresponding to amino acid residue 196 of SEQ ID No. 1 has been substituted with an Arg.

In one embodiment, the amino acid residue in the position corresponding to amino acid residue 197 of SEQ ID No. 1 has been substituted with an Asp.

In one embodiment, a peptide according to the present invention carries a substitution mutation in one or more of the positions corresponding to amino acid residues 31, 68, and 190 of SEQ ID No. 1.

In one embodiment, a peptide according to the present invention carries substitution mutations in the positions corresponding to amino acid residues 31, 68, and 190 of SEQ ID No. 1.

In one embodiment, a peptide according to the present invention carries substitution mutations in the positions corresponding to amino acid residues 31 and 190 of SEQ ID No. 1.

In one embodiment, a peptide according to the present invention carries substitution mutations in the positions corresponding to amino acid residues 68 and 190 of SEQ ID No. 1.

In one embodiment, the amino acid residue in the position corresponding to amino acid residue 31 of SEQ ID No. 1 has been substituted with a Glu.

In one embodiment, the amino acid residue in the position corresponding to amino acid residue 31 of SEQ ID No. 1 has been substituted with an Arg.

In one embodiment, the amino acid residue in the position corresponding to amino acid residue 68 of SEQ ID No. 1 has been substituted with an Asn.

In one embodiment, the amino acid residue in the position corresponding to amino acid residue 190 of SEQ ID No. 1 has been substituted with an Arg.

Mutations in one region of BS1 may be performed as single mutations or in combination with other mutations in the same region or one or more mutations in other regions of BS1.

In one embodiment, peptides according to the present invention also carry one or more substitution mutations in the positions corresponding to amino acid residues 61, 71 and 73 of SEQ ID No. 1 as described in the International patent application PCT/EP2007/060501. In one embodiment, the amino acid residue corresponding to position 71 has been substituted with an alanine. In one embodiment, peptides according to the present invention also carry substitutions in one or more of the positions corresponding to amino acid residues 61 and 73 of SEQ ID No. 1 as described in the International patent application PCT/EP2007/060501. In one embodiment, the amino acid residue in the position corresponding to position 61 of SEQ ID No. 1 has been substituted with an alanine. In one embodiment, the amino acid residue in the position corresponding to position 73 of SEQ ID No. 1 has been substituted with a leucine. In one embodiment, the amino acid residue in the position corresponding to position 73 of SEQ ID No. 1 has been substituted with an alanine.

Mutations as described according to the present invention may be performed in combination with for instance mutations, which gives the peptide antagonistic properties. In one embodiment, peptides according to the present invention, which binds to the human prolactin receptor via binding site 1, also carry substitution mutations, or other mutations or derivatisations, which makes the peptide an antagonist of hPRL-R. Such mutations may for instance be mutations, which disrupt the binding of the peptide to the prolactin receptor via BS2, such as mutations in BS2. Four prolactin receptor antagonists having mutations in BS2 are currently known, namely G129R-hPRL, G129R-hPRL(Δ1-9), and G129R-hPRL (Δ1-14), see Goffin et al. Endocrine Rev. 26, 400-422 (2005)). In one embodiment, peptides according to the present invention, which binds to the human prolactin receptor via binding site 1, also carry a substitution mutation of the amino acid residue in the position corresponding to amino acid residue 129 of SEQ ID No. 1. In one embodiment, the amino acid residue in the position corresponding to amino acid residue 129 of SEQ ID No. 1 has been substituted with an arginine. In one embodiment, peptides according to the present invention, which binds to the human prolactin receptor via binding site 1, also carry a substitution mutation of the amino acid residue in the position corresponding to amino acid residue 179 of SEQ ID No. 1. In one embodiment, the amino acid residue in the position corresponding to amino acid residue 179 of SEQ ID No. 1 has been substituted with an aspartic acid. The amino acid residue in SEQ ID No. 2, which corresponds to amino acid residue 129 in SEQ ID No. 1 is Gly120. In one embodiment, the peptide according to the invention is a variant of human growth hormone as described above, and at least one or more of said antagonistic mutations are selected from G120R or G120K.

The present invention provides an isolated nucleic acid construct encoding a peptide according to the present invention.

As used herein the term “nucleic acid construct” is intended to indicate any nucleic acid molecule of cDNA, genomic DNA, synthetic DNA or RNA origin. The term “construct” is intended to indicate a nucleic acid segment which may be single- or double-stranded, and which may be based on a complete or partial naturally occurring nucleotide sequence encoding a peptide of interest. The construct may optionally contain other nucleic acid segments.

A nucleic acid construct of the invention may suitably be of genomic or cDNA origin, for instance obtained by preparing a genomic or cDNA library and screening for DNA sequences coding for all or part of the peptide by hybridization using synthetic oligonucleotide probes in accordance with standard techniques (cf. J. Sambrook et al, 1989, Molecular Cloning, A Laboratory Manual, 2d edition, Cold Spring Harbor, N.Y.) and by introducing the relevant mutations as it is known in the art. A nucleic acid construct of the invention may also be prepared synthetically by established standard methods, e.g. the phosphoamidite method described by Beaucage and Caruthers, Tetrahedron Letters 22, 1859-1869 (1981), or the method described by Matthes et al., EMBO Journal 3, 801-805 (1984). According to the phosphoamidite method, oligonucleotides are synthesized, e.g. in an automatic DNA synthesizer, purified, annealed, ligated and cloned in suitable vectors. Furthermore, the nucleic acid construct may be of mixed synthetic and genomic, mixed synthetic and cDNA or mixed genomic and cDNA origin prepared by ligating fragments of synthetic, genomic or cDNA origin (as appropriate), the fragments corresponding to various parts of the entire nucleic acid construct, in accordance with standard techniques. The nucleic acid construct may also be prepared by polymerase chain reaction using specific primers, for instance as described in U.S. Pat. No. 4,683,202 or Saiki et al., Science 239, 487-491 (1988). In one embodiment, the nucleic acid construct of the invention is a DNA construct which term will be used exclusively in the following for convenience. The statements in the following may also read on other nucleic acid constructs of the invention with appropriate adaptions as it will be clear for a person skilled in the art.

In one embodiment, the present invention relates to a recombinant vector comprising a DNA construct of the invention. The recombinant vector into which the DNA construct of the invention is inserted may be any vector which may conveniently be subjected to recombinant DNA procedures, and the choice of vector will often depend on the host cell into which it is to be introduced. Thus, the vector may be an autonomously replicating vector, i.e. a vector which exists as an extrachromosomal entity, the replication of which is independent of chromosomal replication, e.g. a plasmid. Alternatively, the vector may be one which, when introduced into a host cell, is integrated into the host cell genome and replicated together with the chromosome(s) into which it has been integrated. The vector may be an expression vector in which the DNA sequence encoding the peptide of the invention is operably linked to additional segments required for transcription of the DNA. In general, the expression vector is derived from plasmid or viral DNA, or may contain elements of both. The term, “operably linked” indicates that the segments are arranged so that they function in concert for their intended purposes, e.g. transcription initiates in a promoter and proceeds through the DNA sequence coding for the peptide. The promoter may be any DNA sequence which shows transcriptional activity in the host cell of choice and may be derived from genes encoding proteins either homologous or heterologous to the host cell. The DNA sequence encoding the peptide of the invention may also, if necessary, be operably connected to a suitable terminator, such as the human growth hormone terminator (Palmiter et al., op. cit.) or (for fungal hosts) the TPI1 (Alber and Kawasaki, op. cit.) or ADH3 (McKnight et al., op. cit.) terminators. The vector may further comprise elements such as polyadenylation signals (e.g. from SV40 or the adenovirus 5 Elb region), transcriptional enhancer sequences (e.g. the SV40 enhancer) and translational enhancer sequences (e.g. the ones encoding adenovirus VA RNAs). The recombinant vector of the invention may further comprise a DNA sequence enabling the vector to replicate in the host cell in question. The vector may also comprise a selectable marker, e.g. a gene the product of which complements a defect in the host cell, such as the gene coding for dihydrofolate reductase (DHFR) or the Schizosaccharomyces pombe TPI gene (described by P. R. Russell, Gene 40, 125-130 (1985)), or one which confers resistance to a drug, e.g. ampicillin, kanamycin, tetracyclin, chloramphenicol, neomycin, hygromycin or methotrexate. For filamentous fungi, selectable markers include amdS, pyrG, argB, niaD and sC. To direct a peptide of the present invention into the secretory pathway of the host cells, a secretory signal sequence (also known as a leader sequence, prepro sequence or pre sequence) may be provided in the recombinant vector. The secretory signal sequence is joined to the DNA sequence encoding the peptide in the correct reading frame. Secretory signal sequences are commonly positioned 5′ to the DNA sequence encoding the peptide. The secretory signal sequence may be that normally associated with the peptide or may be from a gene encoding another secreted protein. The secretory signal sequence may encode any signal peptide which ensures efficient direction of the expressed peptide into the secretory pathway of the cell.

The procedures used to ligate the DNA sequences coding for the present peptide, the promoter and optionally the terminator and/or secretory signal sequence, respectively, and to insert them into suitable vectors containing the information necessary for replication, are well known to persons skilled in the art (cf., for instance, Sambrook et al., op.cit.).

The host cell into which the DNA construct or the recombinant vector of the invention is introduced may be any cell which is capable of producing the present peptide and includes bacteria, yeast, fungi and higher eukaryotic cells as it is well-known in the state of the art. When expressing the peptide in bacteria such as E. coli, the peptide may be retained in the cytoplasm, typically as insoluble granules (known as inclusion bodies), or may be directed to the periplasmic space by a bacterial secretion sequence. In the former case, the cells are lysed and the granules are recovered and denatured after which the peptide is refolded by diluting the denaturing agent. In the latter case, the peptide may be recovered from the periplasmic space by disrupting the cells, e.g. by sonication or osmotic shock, to release the contents of the periplasmic space and recovering the peptide. The transformed or transfected host cell described above is then cultured in a suitable nutrient medium under conditions permitting the expression of the present peptide, after which the resulting peptide is recovered from the culture. The medium used to culture the cells may be any conventional medium suitable for growing the host cells, such as minimal or complex media containing appropriate supplements. Suitable media are available from commercial suppliers or may be prepared according to published recipes (e.g. in catalogues of the American Type Culture Collection). The peptide produced by the cells may then be recovered from the culture medium by conventional procedures including separating the host cells from the medium by centrifugation or filtration, precipitating the proteinaceous components of the supernatant or filtrate by means of a salt, e.g. ammonium sulphate, purification by a variety of chromatographic procedures, e.g. ion exchange chromatography, gelfiltration chromatography, affinity chromatography, or the like, dependent on the type of peptide in question.

Peptides according to the present invention may be used in the treatment of diseases treatable by administration of prolactin recdptor antagonists, such as breast cancer. The term “treatment” and “treating” as used herein means the management and care of a patient for the purpose of combating a condition, such as a disease or a disorder. The term is intended to include the full spectrum of treatments for a given condition from which the patient is suffering, such as administration of the active compound to alleviate the symptoms or complications, to delay the progression of the disease, disorder or condition, to alleviate or relief the symptoms and complications, and/or to cure or eliminate the disease, disorder or condition as well as to prevent the condition, wherein prevention is to be understood as the management and care of a patient for the purpose of combating the disease, condition, or disorder and includes the administration of the active peptides to prevent the onset of the symptoms or complications. The patient to be treated is preferably a mammal, in particular a human being, but it may also include animals, such as dogs, cats, cows, sheep and pigs. It is to be understood, that therapeutic and prophylactic (preventive) regimes represent separate aspects of the present invention.

A “therapeutically effective amount” of a peptide as used herein means an amount sufficient to cure, alleviate or partially arrest the clinical manifestations of a given disease and its complications. An amount adequate to accomplish this is defined as “therapeutically effective amount”. Effective amounts for each purpose will depend on the type and severity of the disease or injury as well as the weight and general state of the subject. It will be understood that determining an appropriate dosage may be achieved using routine experimentation, by constructing a matrix of values and testing different points in the matrix, which is all within the ordinary skills of a trained physician or veterinary.

The present invention provides a pharmaceutical formulation comprising a peptide of the present invention which is present in a concentration from 10⁻¹⁵ mg/ml to 200 mg/ml, such as 10⁻¹⁰ mg/ml-5 mg/ml, and wherein said formulation has a pH from 2.0 to 10.0. Optionally, said formulation may comprise one or more further cancer agents as described above. The formulation may further comprise a buffer system, preservative(s), tonicity agent(s), chelating agent(s), stabilizers and surfactants. In one embodiment of the invention the pharmaceutical formulation is an aqueous formulation, i.e. formulation comprising water. Such formulation is typically a solution or a suspension. In one embodiment of the invention the pharmaceutical formulation is an aqueous solution. The term “aqueous formulation” is defined as a formulation comprising at least 50% w/w water. Likewise, the term “aqueous solution” is defined as a solution comprising at least 50% w/w water, and the term “aqueous suspension” is defined as a suspension comprising at least 50% w/w water.

In one embodiment the pharmaceutical formulation is a freeze-dried formulation, whereto the physician or the patient adds solvents and/or diluents prior to use.

In one embodiment the pharmaceutical formulation is a dried formulation (e.g. freeze-dried or spray-dried) ready for use without any prior dissolution.

In one embodiment the invention relates to a pharmaceutical formulation comprising an aqueous solution of a peptide of the present invention, and a buffer, wherein said peptide is present in a concentration from 0.1-100 mg/ml, and wherein said formulation has a pH from about 2.0 to about 10.0.

In one embodiment, a pharmaceutical formulation according to the invention is a stabilized formulation. The term “stabilized formulation” refers to a formulation with increased physical stability, increased chemical stability or increased physical and chemical stability.

The term “physical stability” of the protein formulation as used herein refers to the tendency of the protein to form biologically inactive and/or insoluble aggregates of the protein as a result of exposure of the protein to thermo-mechanical stresses and/or interaction with interfaces and surfaces that are destabilizing, such as hydrophobic surfaces and interfaces. Physical stability of the aqueous protein formulations is evaluated by means of visual inspection and/or turbidity measurements after exposing the formulation filled in suitable containers (e.g. cartridges or vials) to mechanical/physical stress (e.g. agitation) at different temperatures for various time periods. Visual inspection of the formulations is performed in a sharp focused light with a dark background. The turbidity of the formulation is characterized by a visual score ranking the degree of turbidity for instance on a scale from 0 to 3 (a formulation showing no turbidity corresponds to a visual score 0, and a formulation showing visual turbidity in daylight corresponds to visual score 3). A formulation is classified physical unstable with respect to protein aggregation, when it shows visual turbidity in daylight. Alternatively, the turbidity of the formulation can be evaluated by simple turbidity measurements well-known to the skilled person. Physical stability of the aqueous protein formulations can also be evaluated by using a spectroscopic agent or probe of the conformational status of the protein. The probe is preferably a small molecule that preferentially binds to a non-native conformer of the protein. One example of a small molecular spectroscopic probe of protein structure is Thioflavin T. Thioflavin T is a fluorescent dye that has been widely used for the detection of amyloid fibrils. In the presence of fibrils, and perhaps other protein configurations as well, Thioflavin T gives rise to a new excitation maximum at about 450 nm and enhanced emission at about 482 nm when bound to a fibril protein form. Unbound Thioflavin T is essentially non-fluorescent at the wavelengths.

Other small molecules can be used as probes of the changes in protein structure from native to non-native states. For instance the “hydrophobic patch” probes that bind preferentially to exposed hydrophobic patches of a protein. The hydrophobic patches are generally buried within the tertiary structure of a protein in its native state, but become exposed as a protein begins to unfold or denature. Examples of these small molecular, spectroscopic probes are aromatic, hydrophobic dyes, such as antrhacene, acridine, phenanthroline or the like. Other spectroscopic probes are metal-amino acid complexes, such as cobalt metal complexes of hydrophobic amino acids, such as phenylalanine, leucine, isoleucine, methionine, and valine, or the like.

The term “chemical stability” of the protein formulation as used herein refers to chemical covalent changes in the protein structure leading to formation of chemical degradation products with potential less biological potency and/or potential increased immunogenic properties compared to the native protein structure. Various chemical degradation products can be formed depending on the type and nature of the native protein and the environment to which the protein is exposed. Elimination of chemical degradation can most probably not be completely avoided and increasing amounts of chemical degradation products is often seen during storage and use of the protein formulation as well-known by the person skilled in the art. Most proteins are prone to deamidation, a process in which the side chain amide group in glutaminyl or asparaginyl residues is hydrolysed to form a free carboxylic acid. Other degradations pathways involves formation of high molecular weight transformation products where two or more protein molecules are covalently bound to each other through transamidation and/or disulfide interactions leading to formation of covalently bound dimer, oligomer and polymer degradation products (Stability of Protein Pharmaceuticals, Ahern. T. J. & Manning M. C., Plenum Press, New York 1992). Oxidation (of for instance methionine residues) can be mentioned as another variant of chemical degradation. The chemical stability of the protein formulation can be evaluated by measuring the amount of the chemical degradation products at various time-points after exposure to different environmental conditions (the formation of degradation products can often be accelerated by for instance increasing temperature). The amount of each individual degradation product is often determined by separation of the degradation products depending on molecule size and/or charge using various chromatography techniques (e.g. SEC-HPLC and/or RP-HPLC).

Hence, as outlined above, a “stabilized formulation” refers to a formulation with increased physical stability, increased chemical stability or increased physical and chemical stability. In general, a formulation must be stable during use and storage (in compliance with recommended use and storage conditions) until the expiration date is reached.

In one embodiment of the invention the pharmaceutical formulation comprising the peptide of the present invention is stable for more than 6 weeks of usage and for more than 3 years of storage.

In one embodiment of the invention the pharmaceutical formulation comprising the peptide of the present invention is stable for more than 4 weeks of usage and for more than 3 years of storage.

In one embodiment of the invention the pharmaceutical formulation comprising the peptide of the present invention is stable for more than 4 weeks of usage and for more than two years of storage.

In one embodiment of the invention the pharmaceutical formulation comprising the peptide of the present invention is stable for more than 2 weeks of usage and for more than two years of storage.

In one embodiment of the invention the pH of the formulation is selected from the list consisting of 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, and 10.0. Each one of these specific pH values constitutes alternative embodiments of the invention.

In one embodiment of the invention the buffer is selected from the group consisting of sodium acetate, sodium carbonate, citrate, glycylglycine, histidine, glycine, lysine, arginine, sodium dihydrogen phosphate, disodium hydrogen phosphate, sodium phosphate, and tris(hydroxymethyl)-aminomethan, bicine, tricine, malic acid, succinate, maleic acid, fumaric acid, tartaric acid, aspartic acid or mixtures thereof. Each one of these specific buffers constitutes an alternative embodiment of the invention.

In one embodiment of the invention the formulation further comprises a pharmaceutically acceptable preservative. In one embodiment of the invention the preservative is selected from the group consisting of phenol, o-cresol, m-cresol, p-cresol, methyl p-hydroxybenzoate, propyl p-hydroxybenzoate, 2-phenoxyethanol, butyl p-hydroxybenzoate, 2-phenylethanol, benzyl alcohol, chlorobutanol, and thiomerosal, bronopol, benzoic acid, imidurea, chlorohexidine, sodium dehydroacetate, chlorocresol, ethyl p-hydroxybenzoate, benzethonium chloride, chlorphenesine (3p-chlorphenoxypropane-1,2-diol) or mixtures thereof. In one embodiment of the invention the preservative is present in a concentration from 0.1 mg/ml to 20 mg/ml. In one embodiment of the invention the preservative is present in a concentration from 0.1 mg/ml to 5 mg/ml. In one embodiment of the invention the preservative is present in a concentration from 5 mg/ml to 10 mg/ml. In one embodiment of the invention the preservative is present in a concentration from 10 mg/ml to 20 mg/ml. Each one of these specific preservatives constitutes an alternative embodiment of the invention. The use of a preservative in pharmaceutical formulations is well-known to the skilled person. For convenience reference is made to Remington: The Science and Practice of Pharmacy, 20^{th} edition, 2000.

In one embodiment of the invention the formulation further comprises an isotonic agent. In one embodiment of the invention the isotonic agent is selected from the group consisting of a salt (e.g. sodium chloride), a sugar or sugar alcohol, an amino acid (e.g. L-glycine, L-histidine, arginine, lysine, isoleucine, aspartic acid, tryptophan, threonine), an alditol (e.g. glycerol (glycerine), 1,2-propanediol (propyleneglycol), 1,3-propanediol, 1,3-butanediol) polyethyleneglycol (e.g. PEG400), or mixtures thereof. Any sugar such as mono-, di-, or polysaccharides, or water-soluble glucans, including for example fructose, glucose, mannose, sorbose, xylose, maltose, lactose, sucrose, trehalose, dextran, pullulan, dextrin, cyclodextrin, soluble starch, hydroxyethyl starch and carboxymethylcellulose-Na may be used. In one embodiment the sugar additive is sucrose. Sugar alcohol is defined as a C4-C8 hydrocarbon having at least one —OH group and includes, for example, mannitol, sorbitol, inositol, galactitol, dulcitol, xylitol, and arabitol. In one embodiment the sugar alcohol additive is mannitol. The sugars or sugar alcohols mentioned above may be used individually or in combination. There is no fixed limit to the amount used, as long as the sugar or sugar alcohol is soluble in the liquid preparation and does not adversely effect the stabilizing effects achieved using the methods of the invention. In one embodiment, the sugar or sugar alcohol concentration is between about 1 mg/ml and about 150 mg/ml. In one embodiment of the invention the isotonic agent is present in a concentration from 1 mg/ml to 50 mg/ml. In one embodiment of the invention the isotonic agent is present in a concentration from 1 mg/ml to 7 mg/ml. In one embodiment of the invention the isotonic agent is present in a concentration from 8 mg/ml to 24 mg/ml. In one embodiment of the invention the isotonic agent is present in a concentration from 25 mg/ml to 50 mg/ml. Each one of these specific isotonic agents constitutes an alternative embodiment of the invention. The use of an isotonic agent in pharmaceutical formulations is well-known to the skilled person. For convenience reference is made to Remington: The Science and Practice of Pharmacy, 20^{th} edition, 2000.

In one embodiment of the invention the formulation further comprises a chelating agent. In one embodiment of the invention the chelating agent is selected from salts of ethylenediaminetetraacetic acid (EDTA), citric acid, and aspartic acid, and mixtures thereof.

In one embodiment of the invention the chelating agent is present in a concentration from 0.1 mg/ml to 5 mg/ml. In one embodiment of the invention the chelating agent is present in a concentration from 0.1 mg/ml to 2 mg/ml. In one embodiment of the invention the chelating agent is present in a concentration from 2 mg/ml to 5 mg/ml. Each one of these specific chelating agents constitutes an alternative embodiment of the invention. The use of a chelating agent in pharmaceutical formulations is well-known to the skilled person. For convenience reference is made to Remington: The Science and Practice of Pharmacy, 20^{th} edition, 2000.

In one embodiment of the invention the formulation further comprises a stabilizer. The use of a stabilizer in pharmaceutical formulations is well-known to the skilled person. For convenience reference is made to Remington: The Science and Practice of Pharmacy, 20^{th} edition, 2000.

More particularly, compositions of the invention are stabilized liquid pharmaceutical formulations whose therapeutically active components include a polypeptide that possibly exhibits aggregate formation during storage in liquid pharmaceutical formulations. By “aggregate formation” is intended a physical interaction between the polypeptide molecules that results in formation of oligomers, which may remain soluble, or large visible aggregates that precipitate from the solution. By “during storage” is intended a liquid pharmaceutical composition or formulation once prepared, is not immediately administered to a subject. Rather, following preparation, it is packaged for storage, either in a liquid form, in a frozen state, or in a dried form for later reconstitution into a liquid form or other form suitable for administration to a subject. By “dried form” is intended the liquid pharmaceutical composition or formulation is dried either by freeze drying (i.e., lyophilization; see, for example, Williams and Polli (1984) J. Parenteral Sci. Technol. 38:48-59), spray drying (see Masters (1991) in Spray-Drying Handbook (5th ed; Longman Scientific and Technical, Essez, U.K.), pp. 491-676; Broadhead et al. (1992) Drug Devel. Ind. Pharm. 18:1169-1206; and Mumenthaler et al. (1994) Pharm. Res. 11:12-20), or air drying (Carpenter and Crowe (1988) Cryobiology 25:459-470; and Roser (1991) Biopharm. 4:47-53). Aggregate formation by a polypeptide during storage of a liquid pharmaceutical formulation can adversely affect biological activity of that polypeptide, resulting in loss of therapeutic efficacy of the pharmaceutical formulation. Furthermore, aggregate formation may cause other problems such as blockage of tubing, membranes, or pumps when the polypeptide-containing pharmaceutical formulation is administered using an infusion system.

The pharmaceutical formulations of the invention may further comprise an amount of an amino acid base sufficient to decrease aggregate formation by the polypeptide during storage of the composition. By “amino acid base” is intended an amino acid or a combination of amino acids, where any given amino acid is present either in its free base form or in its salt form. Where a combination of amino acids is used, all of the amino acids may be present in their free base forms, all may be present in their salt forms, or some may be present in their free base forms while others are present in their salt forms. In one embodiment, amino acids to use in preparing the compositions of the invention are those carrying a charged side chain, such as arginine, lysine, aspartic acid, and glutamic acid. Any stereoisomer (i.e., L, D, or mixtures thereof) of a particular amino acid (e.g. glycine, methionine, histidine, imidazole, arginine, lysine, isoleucine, aspartic acid, tryptophan, threonine and mixtures thereof) or combinations of these stereoisomers, may be present in the pharmaceutical formulations of the invention so long as the particular amino acid is present either in its free base form or its salt form. In one embodiment the L-stereoisomer is used. Compositions of the invention may also be formulated with analogues of these amino acids. By “amino acid analogue” is intended a derivative of the naturally occurring amino acid that brings about the desired effect of decreasing aggregate formation by the polypeptide during storage of the liquid pharmaceutical formulations of the invention. Suitable arginine analogues include, for example, aminoguanidine, ornithine and N-monoethyl L-arginine, suitable methionine analogues include ethionine and buthionine and suitable cysteine analogues include S-methyl-L cysteine. As with the other amino acids, the amino acid analogues are incorporated into the compositions in either their free base form or their salt form. In one embodiment of the invention the amino acids or amino acid analogues are used in a concentration, which is sufficient to prevent or delay aggregation of the protein.

In one embodiment of the invention methionine (or other sulphuric amino acids or amino acid analogous) may be added to inhibit oxidation of methionine residues to methionine sulfoxide when the polypeptide acting as the therapeutic agent is a polypeptide comprising at least one methionine residue susceptible to such oxidation. By “inhibit” is intended minimal accumulation of methionine oxidized species over time. Inhibiting methionine oxidation results in greater retention of the polypeptide in its proper molecular form. Any stereoisomer of methionine (L, D, or mixtures thereof) or combinations thereof can be used. The amount to be added should be an amount sufficient to inhibit oxidation of the methionine residues such that the amount of methionine sulfoxide is acceptable to regulatory agencies. Typically, this means that the composition contains no more than about 10% to about 30% methionine sulfoxide. Generally, this can be achieved by adding methionine such that the ratio of methionine added to methionine residues ranges from about 1:1 to about 1000:1, such as 10:1 to about 100:1.

In one embodiment of the invention the formulation further comprises a stabilizer selected from the group of high molecular weight polymers or low molecular compounds. In one embodiment of the invention the stabilizer is selected from polyethylene glycol (e.g. PEG 3350), polyvinyl alcohol (PVA), polyvinylpyrrolidone, carboxy-/hydroxycellulose or derivates thereof (e.g. HPC, HPC-SL, HPC-L and HPMC), cyclodextrins, sulphur-containing substances as monothioglycerol, thioglycolic acid and 2-methylthioethanol, and different salts (e.g. sodium chloride). Each one of these specific stabilizers constitutes an alternative embodiment of the invention.

The pharmaceutical formulations may also comprise additional stabilizing agents, which further enhance stability of a therapeutically active polypeptide therein. Stabilizing agents of particular interest to the present invention include, but are not limited to, methionine and EDTA, which protect the polypeptide against methionine oxidation, and a nonionic surfactant, which protects the polypeptide against aggregation associated with freeze-thawing or mechanical shearing.

In one embodiment of the invention the formulation further comprises a surfactant.

In one embodiment of the invention the surfactant is selected from a detergent, ethoxylated castor oil, polyglycolyzed glycerides, acetylated monoglycerides, sorbitan fatty acid esters, polyoxypropylene-polyoxyethylene block polymers (eg. poloxamers such as Pluronic® F68, poloxamer 188 and 407, Triton X-100), polyoxyethylene sorbitan fatty acid esters, polyoxyethylene and polyethylene derivatives such as alkylated and alkoxylated derivatives (tweens, e.g. Tween-20, Tween-40, Tween-80 and Brij-35), monoglycerides or ethoxylated derivatives thereof, diglycerides or polyoxyethylene derivatives thereof, alcohols, glycerol, lectins and phospholipids (eg. phosphatidyl serine, phosphatidyl choline, phosphatidyl ethanolamine, phosphatidyl inositol, diphosphatidyl glycerol and sphingomyelin), derivates of phospholipids (eg. dipalmitoyl phosphatidic acid) and lysophospholipids (eg. palmitoyl lysophosphatidyl-L-serine and 1-acyl-sn-glycero-3-phosphate esters of ethanolamine, choline, serine or threonine) and alkyl, alkoxyl(alkyl ester), alkoxy(alkyl ether)-derivatives of lysophosphatidyl and phosphatidylcholines, e.g. lauroyl and myristoyl derivatives of lysophosphatidylcholine, dipalmitoylphosphatidylcholine, and modifications of the polar head group, that is cholines, ethanolamines, phosphatidic acid, serines, threonines, glycerol, inositol, and the positively charged DODAC, DOTMA, DCP, BISHOP, lysophosphatidylserine and lysophosphatidylthreonine, and glycerophospholipids (eg. cephalins), glyceroglycolipids (eg. galactopyransoide), sphingoglycolipids (eg. ceramides, gangliosides), dodecylphosphocholine, hen egg lysolecithin, fusidic acid derivatives—(e.g. sodium tauro-dihydrofusidate etc.), long-chain fatty acids and salts thereof C6-C12 (eg. oleic acid and caprylic acid), acylcarnitines and derivatives, N^{α}-acylated derivatives of lysine, arginine or histidine, or side-chain acylated derivatives of lysine or arginine, N^{α}-acylated derivatives of dipeptides comprising any combination of lysine, arginine or histidine and a neutral or acidic amino acid, N^{α}-acylated derivative of a tripeptide comprising any combination of a neutral amino acid and two charged amino acids, DSS (docusate sodium, CAS registry no [577-11-7]), docusate calcium, CAS registry no [128-49-4]), docusate potassium, CAS registry no [7491-09-0]), SDS (sodium dodecyl sulphate or sodium lauryl sulphate), sodium caprylate, cholic acid or derivatives thereof, bile acids and salts thereof and glycine or taurine conjugates, ursodeoxycholic acid, sodium cholate, sodium deoxycholate, sodium taurocholate, sodium glycocholate, N-Hexadecyl-N,N-dimethyl-3-ammonio-1-propanesulfonate, anionic (alkyl-aryl-sulphonates) monovalent surfactants, zwitterionic surfactants (e.g. N-alkyl-N,N-dimethylammonio-1-propanesulfonates, 3-cholamido-1-propyldimethylammonio-1-propanesulfonate, cationic surfactants (quaternary ammonium bases) (e.g. cetyl-trimethylammonium bromide, cetylpyridinium chloride), non-ionic surfactants (eg. Dodecyl β-D-glucopyranoside), poloxamines (eg. Tetronic's), which are tetrafunctional block copolymers derived from sequential addition of propylene oxide and ethylene oxide to ethylenediamine, or the surfactant may be selected from the group of imidazoline derivatives, or mixtures thereof. Each one of these specific surfactants constitutes an alternative embodiment of the invention.

The use of a surfactant in pharmaceutical formulations is well-known to the skilled person. For convenience reference is made to Remington: The Science and Practice of Pharmacy, 20^{th} edition, 2000.

It is possible that other ingredients may be present in the peptide pharmaceutical formulation of the present invention. Such additional ingredients may include wetting agents, emulsifiers, antioxidants, bulking agents, tonicity modifiers, chelating agents, metal ions, oleaginous vehicles, proteins (e.g., human serum albumin, gelatine or proteins) and a zwitterion (e.g., an amino acid such as betaine, taurine, arginine, glycine, lysine and histidine). Such additional ingredients, of course, should not adversely affect the overall stability of the pharmaceutical formulation of the present invention.

Pharmaceutical formulations containing a peptide of the present invention may be administered to a patient in need of such treatment at several sites, for example, at topical sites, for example, skin and mucosal sites, at sites which bypass absorption, for example, administration in an artery, in a vein, in the heart, and at sites which involve absorption, for example, administration in the skin, under the skin, in a muscle or in the abdomen.

Administration of pharmaceutical formulations according to the invention may be through several routes of administration, for example, lingual, sublingual, buccal, in the mouth, oral, in the stomach and intestine, nasal, pulmonary, for example, through the bronchioles and alveoli or a combination thereof, epidermal, dermal, transdermal, vaginal, rectal, ocular, for examples through the conjunctiva, uretal, and parenteral to patients in need of such a treatment.

Compositions of the current invention may be administered in several dosage forms, for example, as solutions, suspensions, emulsions, microemulsions, multiple emulsion, foams, salves, pastes, plasters, ointments, tablets, coated tablets, rinses, capsules, for example, hard gelatine capsules and soft gelatine capsules, suppositories, rectal capsules, drops, gels, sprays, powder, aerosols, inhalants, eye drops, ophthalmic ointments, ophthalmic rinses, vaginal pessaries, vaginal rings, vaginal ointments, injection solution, in situ transforming solutions, for example in situ gelling, in situ setting, in situ precipitating, in situ crystallization, infusion solution, and implants.

Compositions of the invention may further be compounded in, or attached to, for example through covalent, hydrophobic and electrostatic interactions, a drug carrier, drug delivery system and advanced drug delivery system in order to further enhance stability of the peptide of the present invention, increase bioavailability, increase solubility, decrease adverse effects, achieve chronotherapy well known to those skilled in the art, and increase patient compliance or any combination thereof. Examples of carriers, drug delivery systems and advanced drug delivery systems include, but are not limited to, polymers, for example cellulose and derivatives, polysaccharides, for example dextran and derivatives, starch and derivatives, poly(vinyl alcohol), acrylate and methacrylate polymers, polylactic and polyglycolic acid and block co-polymers thereof, polyethylene glycols, carrier proteins, for example albumin, gels, for example, thermogelling systems, for example block co-polymeric systems well known to those skilled in the art, micelles, liposomes, microspheres, nanoparticulates, liquid crystals and dispersions thereof, L2 phase and dispersions there of, well known to those skilled in the art of phase behaviour in lipid-water systems, polymeric micelles, multiple emulsions, self-emulsifying, self-microemulsifying, cyclodextrins and derivatives thereof, and dendrimers.

Compositions of the current invention may be useful in the formulation of solids, semisolids, powder and solutions for pulmonary administration of a peptide of the present invention, using, for example a metered dose inhaler, dry powder inhaler and a nebulizer, all being devices well known to those skilled in the art.

Compositions of the current invention may be useful in the formulation of controlled, sustained, protracting, retarded, and slow release drug delivery systems. More specifically, but not limited to, compositions are useful in formulation of parenteral controlled release and sustained release systems (both systems leading to a many-fold reduction in number of administrations), well known to those skilled in the art. Even more preferably, are controlled release and sustained release systems administered subcutaneous. Without limiting the scope of the invention, examples of useful controlled release system and compositions are hydrogels, oleaginous gels, liquid crystals, polymeric micelles, microspheres, nanoparticles,

Methods to produce controlled release systems useful for compositions of the current invention include, but are not limited to, crystallization, condensation, co-crystallization, precipitation, co-precipitation, emulsification, dispersion, high pressure homogenisation, encapsulation, spray drying, microencapsulating, coacervation, phase separation, solvent evaporation to produce microspheres, extrusion and supercritical fluid processes. General reference is made to Handbook of Pharmaceutical Controlled Release (Wise, D. L., ed. Marcel Dekker, New York, 2000) and Drug and the Pharmaceutical Sciences vol. 99: Protein Formulation and Delivery (MacNally, E. J., ed. Marcel Dekker, New York, 2000).

Parenteral administration may be performed by subcutaneous, intramuscular, intraperitoneal or intravenous injection by means of a syringe, optionally a pen-like syringe. Alternatively, parenteral administration can be performed by means of an infusion pump. A further option is a composition which may be a solution or suspension for the administration of the peptide of the present inventionin the form of a nasal or pulmonal spray. As a still further option, the pharmaceutical formulations containing the peptide of the present invention can also be adapted to transdermal administration, e.g. by needle-free injection or from a patch, optionally an iontophoretic patch, or transmucosal, e.g. buccal, administration.

The following is a list of embodiments of the present invention, which is not be to construed as limiting.

### Embodiment 1

An isolated peptide, which peptide is a variant of a PRL-like cytokine, said variant comprising
(i) one or more amino acid mutations in the region corresponding to amino acid residue 24 to 35 of SEQ ID No. 1 and/or
(ia) one or more amino acid mutations in the region corresponding to amino acid residue 52 to 58 of SEQ ID No. 1 and/or
(ib) one or more amino acid mutations in the region corresponding to amino acid residue 50 to 57 of SEQ ID No. 1 and/or
(ii) one or more amino acid mutations in the region corresponding to amino acid residue 66 to 83 of SEQ ID No. 1 and/or
(iii) one or more amino acid mutations in the region corresponding to amino acid residue 176 to 199 of SEQ ID No. 1 and/or
(iv) an addition of from 1 to 5 amino acid residues to the C-terminal.

### Embodiment 2

An isolated peptide, which peptide is a variant of a PRL-like cytokine, said variant comprising

(i) one or more amino acid mutations in the region corresponding to amino acid residue 24 to 35 of SEQ ID No. 1 and/or
(ia) one or more amino acid mutations in the region corresponding to amino acid residue 52 to 58 of SEQ ID No. 1 and/or
(ib) one or more amino acid mutations in the region corresponding to amino acid residue 50 to 57 of SEQ ID No. 1 and/or
(ii) one or more amino acid mutations in the region corresponding to amino acid residue 66 to 83 of SEQ ID No. 1 and/or
(iii) one or more amino acid mutations in the region corresponding to amino acid residue 176 to 199 of SEQ ID No. 1.

### Embodiment 3

An isolated peptide according to embodiment 1 or embodiment 2, wherein said peptide comprises one or more amino acid mutations in the region corresponding to amino acid residue 52 to 58 of SEQ ID No. 1.

### Embodiment 4

An isolated peptide according to embodiment 1 or embodiment 2, wherein said peptide comprises one or more amino acid mutations in the region corresponding to amino acid residue 50 to 57 of SEQ ID No. 1.

### Embodiment 5

An isolated peptide according to any of embodiments 1 to 4, wherein at least one of the mutation(s) described under (ia) is in the position corresponding to amino acid residue 51 of SEQ ID No. 1.

### Embodiment 6

An isolated peptide according to any of embodiments 1 to 5, wherein at least one of the mutation(s) described under (ia) is in the position corresponding to amino acid residue 55 of SEQ ID No. 1.

### Embodiment 7

An isolated peptide according to embodiment 6, wherein the amino acid residue in the position corresponding to amino acid residue 55 of SEQ ID No. 1 is substituted with an amino acid residue selected from Leu and Val.

### Embodiment 8

An isolated peptide according to any of embodiments 1 to 7, wherein at least one of the mutation(s) described under (ia) is in the position corresponding to amino acid residue 56 of SEQ ID No. 1.

### Embodiment 9

An isolated peptide according to embodiment 8, wherein the amino acid residue in the position corresponding to amino acid residue 56 of SEQ ID No. 1 is substituted with Gln.

### Embodiment 10

An isolated peptide according to any of embodiments 1 to 9, wherein at least one of the mutation(s) described under (ia) is in the position corresponding to amino acid residue 57 of SEQ ID No. 1.

### Embodiment 11

An isolated peptide, which peptide is a variant of a PRL-like cytokine, said variant comprising
(i) one or more amino acid mutations in the region corresponding to amino acid residue 24 to 35 of SEQ ID No. 1 and/or
(ii) one or more amino acid mutations in the region corresponding to amino acid residue 66 to 83 of SEQ ID No. 1 and/or
(iii) one or more amino acid mutations in the region corresponding to amino acid residue 176 to 199 of SEQ ID No. 1.

### Embodiment 12

An isolated peptide according to any of embodiments 1 to 11, wherein said peptide binds the prolactin receptor.

### Embodiment 13

An isolated peptide according to embodiment 12, wherein the binding of the peptide to the prolactin receptor is determined as described in Assay (I).

### Embodiment 14

An isolated peptide according to any of embodiments 1 to 13, wherein the PRL-like cytokine has at least 80%, such as at least 85%, for instance 90%, such as 95%, for instance 96%, such as 97%, for instance 98%, such as 99% identity to the amino acid sequence of human prolactin, growth hormone, placenta lactogen, interleukin-2, interleukin-3, interleukin-4, interleukin-6, interleukin-17, interleukin-20, interleukin-21, interleukin-31, interleukin-32 or erythropoietin (EPO).

### Embodiment 15

An isolated peptide according to embodiment 14, wherein the PRL-like cytokine is interleukin-2.

### Embodiment 16

An isolated peptide according to embodiment 14, wherein the PRL-like cytokine is interleukin-3.

### Embodiment 17

An isolated peptide according to embodiment 14, wherein the PRL-like cytokine is interleukin-4.

### Embodiment 18

An isolated peptide according to embodiment 14, wherein the PRL-like cytokine is interleukin-6.

### Embodiment 19

An isolated peptide according to embodiment 14, wherein the PRL-like cytokine is interleukin-17.

### Embodiment 20

An isolated peptide according to embodiment 14, wherein the PRL-like cytokine is interleukin-20.

### Embodiment 21

An isolated peptide according to embodiment 14, wherein the PRL-like cytokine is interleukin-21.

### Embodiment 22

An isolated peptide according to embodiment 14, wherein the PRL-like cytokine is interleukin-31.

### Embodiment 23

An isolated peptide according to embodiment 14, wherein the PRL-like cytokine is interleukin-32.

### Embodiment 24

An isolated peptide according to embodiment 14, wherein the PRL-like cytokine is erythropoietin (EPO).

### Embodiment 25

An isolated peptide according to any of embodiments 1 to 14, wherein the PRL-like cytokine has at least 80% identity to SEQ ID No. 1.

### Embodiment 26

An isolated peptide according to embodiment 25, wherein the PRL-like cytokine has at least 85% identity to SEQ ID No. 1.

### Embodiment 27

An isolated peptide according to embodiment 26, wherein the PRL-like cytokine has at least 90% identity to SEQ ID No. 1.

### Embodiment 28

An isolated peptide according to embodiment 27, wherein the PRL-like cytokine has at least 95% identity to SEQ ID No. 1.

### Embodiment 29

An isolated peptide according to embodiment 28, wherein the PRL-like cytokine has at least 96% identity to SEQ ID No. 1.

### Embodiment 30

An isolated peptide according to embodiment 29, wherein the PRL-like cytokine has at least 97% identity to SEQ ID No. 1.

### Embodiment 31

An isolated peptide according to embodiment 30, wherein the PRL-like cytokine has at least 98% identity to SEQ ID No. 1.

### Embodiment 32

An isolated peptide according to embodiment 31, wherein the PRL-like cytokine has at least 99% identity to SEQ ID No. 1.

### Embodiment 33

An isolated peptide according to any of embodiments 25 to 32, wherein the PRL-like cytokine comprises the amino acid sequence of SEQ ID No. 1.

### Embodiment 34

An isolated peptide according to any of embodiments 1 to 13, wherein the PRL-like cytokine has at least 80% identity to SEQ ID No. 2.

### Embodiment 35

An isolated peptide according to embodiment 34, wherein the PRL-like cytokine has at least 85% identity to SEQ ID No. 2.

### Embodiment 36

An isolated peptide according to embodiment 35, wherein the PRL-like cytokine has at least 90% identity to SEQ ID No. 2.

### Embodiment 37

An isolated peptide according to embodiment 36, wherein the PRL-like cytokine has at least 95% identity to SEQ ID No. 2.

### Embodiment 38

An isolated peptide according to embodiment 37, wherein the PRL-like cytokine has at least 96% identity to SEQ ID No. 2.

### Embodiment 39

An isolated peptide according to embodiment 38, wherein the PRL-like cytokine has at least 97% identity to SEQ ID No. 2.

### Embodiment 40

An isolated peptide according to embodiment 39, wherein the PRL-like cytokine has at least 98% identity to SEQ ID No. 2.

### Embodiment 41

An isolated peptide according to embodiment 40, wherein the PRL-like cytokine has at least 99% identity to SEQ ID No. 2.

### Embodiment 42

An isolated peptide according to any of embodiments 34 to 41, wherein the PRL-like cytokine comprises the amino acid sequence of SEQ ID No. 2.

### Embodiment 43

An isolated peptide according to any of embodiments 1 to 13, wherein the PRL-like cytokine has at least 80% identity to SEQ ID No. 3.

### Embodiment 44

An isolated peptide according to embodiment 43, wherein the PRL-like cytokine has at least 85% identity to SEQ ID No. 3.

### Embodiment 45

An isolated peptide according to embodiment 44, wherein the PRL-like cytokine has at least 90% identity to SEQ ID No. 3.

### Embodiment 46

An isolated peptide according to embodiment 45, wherein the PRL-like cytokine has at least 95% identity to SEQ ID No. 3.

### Embodiment 47

An isolated peptide according to embodiment 46, wherein the PRL-like cytokine has at least 96% identity to SEQ ID No. 3.

### Embodiment 48

An isolated peptide according to embodiment 47, wherein the PRL-like cytokine has at least 97% identity to SEQ ID No. 3.

### Embodiment 49

An isolated peptide according to embodiment 48, wherein the PRL-like cytokine has at least 98% identity to SEQ ID No. 3.

### Embodiment 50

An isolated peptide according to embodiment 49, wherein the PRL-like cytokine has at least 99% identity to SEQ ID No. 3.

### Embodiment 51

An isolated peptide according to any of embodiments 43 to 50, wherein the PRL-like cytokine comprises the amino acid sequence of SEQ ID No. 3.

### Embodiment 52

An isolated peptide according to any of embodiments 1 to 51, wherein said peptide comprises one or more amino acid mutations in the region corresponding to amino acid residues 24 to 35 of SEQ ID No. 1.

### Embodiment 53

An isolated peptide according to any of embodiments 1 to 52, wherein at least one of the mutation(s) described under (i) is in the position corresponding to amino acid residue 25 of SEQ ID No. 1.

### Embodiment 54

An isolated peptide according to embodiment 53, wherein the amino acid residue in the position corresponding to amino acid residue 25 of SEQ ID No. 1 is substituted with a Gln.

### Embodiment 55

An isolated peptide according to any of embodiments 1 to 54, wherein the mutation(s) described under (i) is in the region corresponding to amino acid residue 26 to 33 of SEQ ID No. 1.

### Embodiment 56

An isolated peptide according to any of embodiments 1 to 55, wherein at least one of the mutation(s) described under (i) is in the position corresponding to amino acid residue 28 of SEQ ID No. 1.

### Embodiment 57

An isolated peptide according to embodiment 56, wherein the amino acid residue in the position corresponding to amino acid residue 28 of SEQ ID No. 1 is substituted with an Asn.

### Embodiment 58

An isolated peptide according to any of embodiments 1 to 57, wherein at least one of the mutation(s) described under (i) is a substitution in the position corresponding to amino acid residue 31 or a substitution in the position corresponding to amino acid residue 33 of SEQ ID No. 1.

### Embodiment 59

An isolated peptide according to any of embodiments 1 to 58, wherein at least one of the mutation(s) described under (i) is in the position corresponding to amino acid residue 31 of SEQ ID No. 1.

### Embodiment 60

An isolated peptide according to embodiment 59, wherein the amino acid residue in the position corresponding to amino acid residue 31 of SEQ ID No. 1 is substituted with an Arg.

### Embodiment 61

An isolated peptide according to embodiment 59, wherein the amino acid residue in the position corresponding to amino acid residue 31 of SEQ ID No. 1 is substituted with a Glu.

### Embodiment 62

An isolated peptide according to embodiment 59, wherein the amino acid residue in the position corresponding to amino acid residue 31 of SEQ ID No. 1 is substituted with a Ser.

### Embodiment 63

An isolated peptide according to any of embodiments 1 to 62, wherein at least one of the mutation(s) described under (i) is in the position corresponding to amino acid residue 33 of SEQ ID No. 1.

### Embodiment 64

An isolated peptide according to embodiment 63, wherein the amino acid residue in the position corresponding to amino acid residue 33 of SEQ ID No. 1 is substituted with an Asp.

### Embodiment 65

An isolated peptide according to embodiment 63, wherein the amino acid residue in the position corresponding to amino acid residue 33 of SEQ ID No. 1 is substituted with an Ala.

### Embodiment 66

An isolated peptide according to any of embodiments 1 to 65, wherein the mutation(s) described under (i) is not in the amino acid residue corresponding to amino acid residue 30 of SEQ ID No. 1.

### Embodiment 67

An isolated peptide according to any of embodiments 1 to 66, wherein said peptide comprises one or more amino acid mutations in the region corresponding to amino acid residues 66 to 83 of SEQ ID No. 1.

### Embodiment 68

An isolated peptide according to any of embodiments 1 to 67, wherein said peptide comprises one or more amino acid mutations in the region corresponding to amino acid residue 67 to 83 of SEQ ID No. 1.

### Embodiment 69

An isolated peptide according to any of embodiments 1 to 68, wherein the mutation(s) described under (ii) is not in the amino acid residue corresponding to amino acid residue 69 of SEQ ID No. 1.

### Embodiment 70

An isolated peptide according to any of embodiments 1 to 69, wherein at least one of the mutation(s) described under (ii) is in the position corresponding to amino acid residue 70 of SEQ ID No. 1.

### Embodiment 71

An isolated peptide according to embodiment 70, wherein the amino acid residue in the position corresponding to amino acid residue 70 of SEQ ID No. 1 is substituted with a Lys.

### Embodiment 72

An isolated peptide according to any of embodiments 67 to 71, wherein any substitution in the position corresponding to amino acid residue 73 of SEQ ID No. 1 is not a substitution with alanine.

### Embodiment 73

An isolated peptide according to embodiment 72, wherein any substitution in the position corresponding to amino acid residue 73 of SEQ ID No. 1 is a substitution with a leucine.

### Embodiment 74

An isolated peptide according to embodiment 72, wherein the peptide is not mutated in the position corresponding to amino acid residue 73 of SEQ ID No. 1.

### Embodiment 75

An isolated peptide according to any of embodiments 1 to 74, wherein at least one of the mutation(s) described under (ii) is a substitution in the position corresponding to amino acid residue 68 or a substitution in the position corresponding to amino acid residue 75 or a substitution in the position corresponding to amino acid residue 76 or a substitution in the position corresponding to amino acid residue 80 of SEQ ID No. 1.

### Embodiment 76

An isolated peptide according to any of embodiments 1 to 75, wherein at least one of the mutation(s) described under (ii) is in the position corresponding to amino acid residue 68 of SEQ ID No. 1.

### Embodiment 77

An isolated peptide according to embodiment 76, wherein the amino acid residue in the position corresponding to amino acid residue 68 of SEQ ID No. 1 is substituted with an Asn.

### Embodiment 78

An isolated peptide according to any of embodiments 1 to 77, wherein at least one of the mutation(s) described under (ii) is in the position corresponding to amino acid residue 75 of SEQ ID No. 1.

### Embodiment 79

An isolated peptide according to embodiment 78, wherein the amino acid residue in the position corresponding to amino acid residue 75 of SEQ ID No. 1 is substituted with a Thr.

### Embodiment 80

An isolated peptide according to any of embodiments 1 to 79, wherein at least one of the mutation(s) described under (ii) is in the position corresponding to amino acid residue 76 of SEQ ID No. 1.

### Embodiment 81

An isolated peptide according to embodiment 80, wherein the amino acid residue in the position corresponding to amino acid residue 76 of SEQ ID No. 1 is substituted with a Ser.

### Embodiment 82

An isolated peptide according to any of embodiments 1 to 81, wherein at least one of the mutation(s) described under (ii) is in the position corresponding to amino acid residue 80 of SEQ ID No. 1.

### Embodiment 83

An isolated peptide according to embodiment 82, wherein the amino acid residue in the position corresponding to amino acid residue 80 of SEQ ID No. 1 is substituted with a Leu.

### Embodiment 84

An isolated peptide according to any of embodiments 1 to 83, wherein said peptide comprises one or more amino acid mutations in the region corresponding to amino acid residues 176 to 199 of SEQ ID No. 1.

### Embodiment 85

An isolated peptide according to any of embodiments 1 to 84, wherein the mutation(s) described under (iii) is not in the amino acid residue corresponding to amino acid residue 176 of SEQ ID No. 1.

### Embodiment 86

An isolated peptide according to any of embodiments 1 to 85, wherein the mutation(s) described under (iii) is not in the amino acid residue corresponding to amino acid residue 177 of SEQ ID No. 1.

### Embodiment 87

An isolated peptide according to any of embodiments 1 to 86, wherein the mutation(s) described under (iii) is not in the amino acid residue corresponding to amino acid residue 180 of SEQ ID No. 1.

### Embodiment 88

An isolated peptide according to any of embodiments 1 to 87, wherein the mutation(s) described under (iii) is not in the amino acid residue corresponding to amino acid residue 181 of SEQ ID No. 1.

### Embodiment 89

An isolated peptide according to any of embodiments 1 to 88, wherein the mutation(s) described under (iii) is not in the amino acid residue corresponding to amino acid residue 185 of SEQ ID No. 1.

### Embodiment 90

An isolated peptide according to any of embodiments 1 to 89, wherein the mutation(s) described under (iii) is not in the amino acid residue corresponding to amino acid residue 187 of SEQ ID No. 1.

### Embodiment 91

An isolated peptide according to any of embodiments 1 to 90, wherein at least one of the mutation(s) described under (iii) is in the position corresponding to amino acid residue 182 of SEQ ID No. 1.

### Embodiment 92

An isolated peptide according to embodiment 91, wherein the amino acid residue in the position corresponding to amino acid residue 182 of SEQ ID No. 1 is substituted with a Val.

### Embodiment 93

An isolated peptide according to any of embodiments 1 to 92, wherein at least one of the mutation(s) described under (iii) is in the region corresponding to amino acid residue 188 to 199 of SEQ ID No. 1.

### Embodiment 94

An isolated peptide according to any of embodiments 1 to 93, wherein at least one of the mutation(s) described under (iii) is in the position corresponding to amino acid residue 190 of SEQ ID No. 1.

### Embodiment 95

An isolated peptide according to embodiment 94, wherein the amino acid residue in the position corresponding to amino acid residue 190 of SEQ ID No. 1 is substituted with an Arg.

### Embodiment 96

An isolated peptide according to any of embodiments 1 to 95, wherein at least one of the mutation(s) described under (iii) is in the position corresponding to amino acid residue 194 of SEQ ID No. 1.

### Embodiment 97

An isolated peptide according to embodiment 96, wherein the amino acid residue in the position corresponding to amino acid residue 194 of SEQ ID No. 1 is substituted with a Val.

### Embodiment 98

An isolated peptide according to any of embodiments 1 to 97, wherein at least one of the mutation(s) described under (iii) is in the position corresponding to amino acid residue 195 of SEQ ID No. 1.

### Embodiment 99

An isolated peptide according to embodiment 98, wherein the amino acid residue in the position corresponding to amino acid residue 195 of SEQ ID No. 1 is substituted with a Tyr.

### Embodiment 100

An isolated peptide according to any of embodiments 1 to 99, wherein at least one of the mutation(s) described under (iii) is in the position corresponding to amino acid residue 196 of SEQ ID No. 1.

### Embodiment 101

An isolated peptide according to embodiment 100, wherein the amino acid residue in the position corresponding to amino acid residue 196 of SEQ ID No. 1 is substituted with an Arg.

### Embodiment 102

An isolated peptide according to any of embodiments 1 to 101, wherein at least one of the mutation(s) described under (iii) is in the position corresponding to amino acid residue 197 of SEQ ID No. 1.

### Embodiment 103

An isolated peptide according to embodiment 102, wherein the amino acid residue in the position corresponding to amino acid residue 197 of SEQ ID No. 1 is substituted with an Arg.

### Embodiment 104

An isolated peptide according to any of embodiments 96 to 103, wherein said peptide carries substitution mutations in the position corresponding to amino acid residues 194, 195, 196 and 197 of SEQ ID No. 1.

### Embodiment 105

An isolated peptide according to embodiment 104, wherein the amino acid residue in the position corresponding to amino acid residue 194 of SEQ ID No. 1 is substituted with a Val, the amino acid residue in the position corresponding to amino acid residue 195 of SEQ ID No. 1 is substituted with a Tyr, the amino acid residue in the position corresponding to amino acid residue 196 of SEQ ID No. 1 is substituted with an Arg, and the amino acid residue in the position corresponding to amino acid residue 197 of SEQ ID No. 1 is substituted with an Arg.

### Embodiment 106

An isolated peptide, which peptide is a variant of a PRL-like cytokine, said variant comprising one or more amino acid mutations, which stabilizes the structure of the prolactin molecule.

### Embodiment 107

An isolated peptide according to embodiment 106, wherein said peptide binds the prolactin receptor.

### Embodiment 108

An isolated peptide according to embodiment 107, wherein the binding of the peptide to the prolactin receptor is determined as described in Assay (I) or Assay (II) or Assay (III) as described herein.

### Embodiment 109

An isolated peptide according to any of embodiments 106 to 108, wherein the PRL-like cytokine has at least 80% identity to SEQ ID No. 1.

### Embodiment 110

An isolated peptide according to embodiment 109, wherein the PRL-like cytokine has at least 85% identity to SEQ ID No. 1.

### Embodiment 111

An isolated peptide according to embodiment 110, wherein the PRL-like cytokine has at least 90% identity to SEQ ID No. 1.

### Embodiment 112

An isolated peptide according to embodiment 111, wherein the PRL-like cytokine has at least 95% identity to SEQ ID No. 1.

### Embodiment 113

An isolated peptide according to embodiment 112, wherein the PRL-like cytokine has at least 96% identity to SEQ ID No. 1.

### Embodiment 114

An isolated peptide according to embodiment 113, wherein the PRL-like cytokine has at least 97% identity to SEQ ID No. 1.

### Embodiment 115

An isolated peptide according to embodiment 114, wherein the PRL-like cytokine has at least 98% identity to SEQ ID No. 1.

### Embodiment 116

An isolated peptide according to embodiment 115, wherein the PRL-like cytokine has at least 99% identity to SEQ ID No. 1.

### Embodiment 117

An isolated peptide according to any of embodiments 109 to 116, wherein the PRL-like cytokine comprises the amino acid sequence of SEQ ID No. 1.

### Embodiment 118

An isolated peptide according to any of embodiments 106 to 108, wherein the PRL-like cytokine has at least 80% identity to SEQ ID No. 2.

### Embodiment 119

An isolated peptide according to embodiment 118, wherein the PRL-like cytokine has at least 85% identity to SEQ ID No. 2.

### Embodiment 120

An isolated peptide according to embodiment 119, wherein the PRL-like cytokine has at least 90% identity to SEQ ID No. 2.

### Embodiment 121

An isolated peptide according to embodiment 120, wherein the PRL-like cytokine has at least 95% identity to SEQ ID No. 2.

### Embodiment 122

An isolated peptide according to embodiment 121, wherein the PRL-like cytokine has at least 96% identity to SEQ ID No. 2.

### Embodiment 123

An isolated peptide according to embodiment 122, wherein the PRL-like cytokine has at least 97% identity to SEQ ID No. 2.

### Embodiment 124

An isolated peptide according to embodiment 123, wherein the PRL-like cytokine has at least 98% identity to SEQ ID No. 2.

### Embodiment 125

An isolated peptide according to embodiment 124, wherein the PRL-like cytokine has at least 99% identity to SEQ ID No. 2.

### Embodiment 126

An isolated peptide according to any of embodiments 118 to 125, wherein the PRL-like cytokine comprises the amino acid sequence of SEQ ID No. 2.

### Embodiment 127

An isolated peptide according to any of embodiments 106 to 108, wherein the PRL-like cytokine has at least 80% identity to SEQ ID No. 3.

### Embodiment 128

An isolated peptide according to embodiment 127, wherein the PRL-like cytokine has at least 85% identity to SEQ ID No. 3.

### Embodiment 129

An isolated peptide according to embodiment 128, wherein the PRL-like cytokine has at least 90% identity to SEQ ID No. 3.

### Embodiment 130

An isolated peptide according to embodiment 129, wherein the PRL-like cytokine has at least 95% identity to SEQ ID No. 3.

### Embodiment 131

An isolated peptide according to embodiment 130, wherein the PRL-like cytokine has at least 96% identity to SEQ ID No. 3.

### Embodiment 132

An isolated peptide according to embodiment 131, wherein the PRL-like cytokine has at least 97% identity to SEQ ID No. 3.

### Embodiment 133

An isolated peptide according to embodiment 132, wherein the PRL-like cytokine has at least 98% identity to SEQ ID No. 3.

### Embodiment 134

An isolated peptide according to embodiment 133, wherein the PRL-like cytokine has at least 99% identity to SEQ ID No. 3.

### Embodiment 135

An isolated peptide according to any of embodiments 127 to 134, wherein the PRL-like cytokine comprises the amino acid sequence of SEQ ID No. 3.

### Embodiment 136

An isolated peptide according to any of embodiments 1 to 105, wherein said peptide comprises one or more amino acid mutations, which stabilizes the secondary structure of the prolactin molecule.

### Embodiment 137

An isolated peptide according to any of embodiments 106 to 136, wherein the stabilization of PRL is determined by use of HX-MS technology as described in Example 1.

### Embodiment 138

An isolated peptide according to any of embodiments 106 to 137, wherein one or more of said amino acid mutation(s) stabilizes the 4-helix bundle structure in prolactin.

### Embodiment 139

An isolated peptide according to any of embodiments 106 to 138, wherein one or more of said amino acid mutation(s) improves the helix capping in helix 1, helix 2, helix 3 and/or helix 4 of PRL.

### Embodiment 140

An isolated peptide according to any of embodiments 106 to 139, wherein one or more of said amino acid mutations are selected from mutations in the amino acid residues corresponding to Ala-111 and Glu-162.

### Embodiment 141

An isolated peptide according to embodiment 140, wherein the amino acid residue corresponding to Ala-111 is substituted with Asp, Asn, Ser or Thr.

### Embodiment 142

An isolated peptide according to embodiment 140 or embodiment 141, wherein the amino acid residue corresponding to Glu-162 is substituted with Asp.

### Embodiment 143

An isolated peptide according to any of embodiments 106 to 142, wherein one or more of said amino acid mutation(s) introduces salt bridges in helical segments exposed to solvent.

### Embodiment 144

An isolated peptide according to any of embodiments 106 to 143, wherein one of said amino acid mutations is a mutation in the amino acid residue corresponding to Asn-92.

### Embodiment 145

An isolated peptide according to embodiment 144, wherein the amino acid residue corresponding to Asn-92 is substituted with Asp.

### Embodiment 146

An isolated peptide according to any of embodiments 106 to 145, wherein two or more of said amino acid mutation(s) introduces non-native disulfide bonds into prolactin.

### Embodiment 147

An isolated peptide according to embodiment 146, wherein said two amino acid mutations are selected from mutations in the positions corresponding to L1C/S135C, A22C/G129C, V23C/L186C, S26C/D183C, L32C/I119C, S33C/L175C, S33C/R176C, S33C/S179C, M36C/K115C, F37C/L172C, T45C/151C, S57C/N170C, H59C/P148C, L63C/S86C, P66C/Q71C, P66C/A72C, Q77C/V137C, K78C/K142C, K78C/H138C, L81C/V134C, S82C/E143C, S82C/N144C, V85C/N144C, S86C/I146C, L88C/L127C, R89C/Y147C, S90C/Y147C, E93C/W150C, L95C/E120C, V99C/A116C, V102C/L113C, M105C/A108C, H138C/T141C, M158C/R164C, and D160C/S193C.

### Embodiment 148

An isolated peptide according to any of embodiments 106 to 147, wherein one or more of said amino acid mutation(s) is a substitution of a solvent exposed hydrophobic residue with a polar residue.

### Embodiment 149

An isolated peptide according to embodiment 148, wherein one or more of said amino acid mutations are selected from mutations in the amino acid residues corresponding to Ile-146 and Val-149.

### Embodiment 150

An isolated peptide according to embodiment 149, wherein the amino acid residue corresponding to Ile-146 is substituted with serine or threonine.

### Embodiment 151

An isolated peptide according to embodiment 149 or embodiment 150, wherein the amino acid residue corresponding to Val-149 is substituted with serine or threonine.

### Embodiment 152

An isolated peptide according to any of embodiments 106 to 151, wherein one or more of said amino acid mutation(s) improves the packing interactions at the hydrophobic core of the 4-helix bundle structure.

### Embodiment 153

An isolated peptide according to embodiment 152, wherein one or more of said amino acid mutations are selected from mutations in the amino acid residues corresponding to Leu-95, Ile-119 and Leu-175.

### Embodiment 154

An isolated peptide according to embodiment 153, wherein the amino acid residue corresponding to Leu-95 is substituted with Val.

### Embodiment 155

An isolated peptide according to embodiment 153 or embodiment 154, wherein the amino acid residue corresponding to Ile-119 is substituted with Val.

### Embodiment 156

An isolated peptide according to any of embodiments 153 to 155, wherein the amino acid residue corresponding to Leu-175 is substituted with Pro.

### Embodiment 157

An isolated peptide according to any of embodiments 1 to 156, wherein said peptide is also mutated in one or more positions corresponding to amino acid residues 20 to 36 and/or 40 to 63 and/or 173 to 185 of SEQ ID No. 1.

### Embodiment 158

An isolated peptide according to any of embodiments 1 to 157, wherein said peptide has an increased affinity to the prolactin receptor as compared to human prolactin.

### Embodiment 159

An isolated peptide according to embodiment 158, wherein the affinity to the prolactin receptor is determined according to Assay (I) as described herein.

### Embodiment 160

An isolated peptide according to any of embodiments 1 to 159, wherein the binding of said peptide for the prolactin receptor has a dissociation konstant (K_{d}) at least three times less than that of wildtype human PRL binding to the prolactin receptor.

### Embodiment 161

An isolated peptide according to any of embodiments 1 to 160, wherein said peptide is capable of binding to the human growth hormone receptor.

### Embodiment 162

An isolated peptide according to embodiment 161, wherein the binding to the human growth hormone receptor is determined by use of the assay as described as Assay (I) herein.

### Embodiment 163

An isolated peptide according to any of embodiments 1 to 162 also comprising at least one amino acid substitution selected from an amino acid mutation in the position corresponding to position 61, an amino acid mutation in the position corresponding to position 71 and an amino acid mutation in the position corresponding to position 73 of SEQ ID No. 1.

### Embodiment 164

An isolated peptide according to embodiment 163 having an amino acid mutation in the position corresponding to position 71 of SEQ ID No. 1.

### Embodiment 165. 6

An isolated peptide according to embodiment 164, wherein the amino acid residue in the position corresponding to position 71 of SEQ ID No. 1 has been substituted with an alanine.

### Embodiment 166

An isolated peptide according to any of embodiments 1 to 165 also comprising at least one amino acid substitution selected from an amino acid mutation in the position corresponding to position 61 and an amino acid mutation in the position corresponding to position 73 of SEQ ID No. 1.

### Embodiment 167

An isolated peptide according to any of embodiments 163 to 166 having an amino acid mutation in the position corresponding to position 61 of SEQ ID No. 1.

### Embodiment 168

An isolated peptide according to embodiment 167, wherein the amino acid residue in the position corresponding to position 61 of SEQ ID No. 1 has been substituted with an alanine.

### Embodiment 169

An isolated peptide according to any of embodiments 163 to 168 having an amino acid mutation in the position corresponding to position 73 of SEQ ID No. 1.

### Embodiment 170

An isolated peptide according to embodiment 169, wherein the amino acid residue in the position corresponding to position 73 of SEQ ID No. 1 has been substituted with a leucine.

### Embodiment 171

An isolated peptide according to embodiment 169, wherein the amino acid residue in the position corresponding to position 73 of SEQ ID No. 1 has been substituted with an alanine.

### Embodiment 172

An isolated peptide according to any of embodiments 1 to 171, which peptide have been modified so that binding of the peptide via BS2 to the prolactin receptor is disrupted.

### Embodiment 173

An isolated peptide according to embodiment 172, wherein said disruption is determined by use of the assay described in Assay II or Assay III or Assay IV.

### Embodiment 174

An isolated peptide according to embodiment 172 or embodiment 173, wherein said disruption is achieved by introducing one or more mutations into BS2 to prevent or reduce interaction of BS2 with PRL-R.

### Embodiment 175

An isolated peptide according to any of embodiments 172 to 174, wherein at least one of said disruptive mutations is a mutation in the amino acid residue corresponding to Gly-129 in SEQ ID No. 1.

### Embodiment 176

An isolated peptide according to embodiment 175, wherein the amino acid residue corresponding to Gly-129 in SEQ ID No. 1 has been substituted with an Arg.

### Embodiment 177

An isolated peptide according to any of embodiments 1 to 176, wherein the amino acid residues corresponding to positions 1 to 9 in PRL have been deleted.

### Embodiment 178

An isolated peptide according to embodiment 177, wherein the amino acid residues corresponding to positions 1 to 14 in PRL have been deleted.

### Embodiment 179

An isolated peptide according to any of embodiments 1 to 178, which is an antagonist of the prolactin receptor.

### Embodiment 180

An isolated peptide according to embodiment 179, wherein said antagonism is determined using Assay (II) as described herein.

### Embodiment 181

An isolated nucleic acid encoding a peptide according to any of embodiments 1 to 180.

### Embodiment 182

A vector comprising a nucleic acid construct according to embodiment 181.

### Embodiment 183

A host cell comprising a nucleic acid construct of embodiment 181, or a vector of embodiment 182.

### Embodiment 184

An antibody that specifically binds a peptide according to any of embodiments 1 to 180.

### Embodiment 185

An antibody according to embodiment 184, which antibody does not bind to a peptide comprising the amino acid sequence of SEQ ID No. 1, SEQ ID No. 2 or SEQ ID No. 3.

### Embodiment 186

A pharmaceutical formulation comprising a peptide according to any of embodiments 1 to 180.

### Embodiment 187

A peptide according to any of embodiments 1 to 180 for use in therapy.

### Embodiment 188

A peptide according to embodiment 187 for use in treating or preventing a proliferative disorder.

### Embodiment 189

A peptide according to embodiment 188, wherein said proliferative disorder is a cancer.

### Embodiment 190

A peptide according to embodiment 189, wherein said cancer is selected from an estrogen dependent cancer, breast cancer, prostate cancer, lung cancer, colorectal cancer, head and neck cancer, ovarian cancer, cervical cancer, bladder cancer, pancreatic cancer, gastrointestinal cancer, leukaemia, skin cancer, and lymphoma.

### Embodiment 191

A peptide according to embodiment 190, wherein said cancer is breast, prostate, colorectal, head and neck or lung cancer.

### Embodiment 192

A peptide according to embodiment 191, wherein said cancer is breast cancer.

### Embodiment 193

A peptide according to any of embodiments 187 to 192 for use alone or in combination with anti-estrogen therapies.

### Embodiment 194

A peptide according to any of embodiments 187 to 192 for use alone or in combination with inhibitors of growth factor receptors signalling.

### Embodiment 195

A peptide according to any of embodiments 187 to 192 for use alone or in combination with anti-angiogenesis therapies.

### Embodiment 196

A peptide according to any of embodiments 187 to 192 for use alone or in combination with anti-lymphogenic therapies.

### Embodiment 197

A peptide according to any of embodiments 187 to 192 for use alone or in combination with immunomodulating therapies.

### Embodiment 198

A peptide according to any of embodiments 187 to 192 for use alone or in combination with chemotherapeutic agents.

### Embodiment 199

A pharmaceutical formulation comprising a peptide according to any of embodiments 1 to 180.

### Embodiment 200

A pharmaceutical formulation according to embodiment 199 for use in the treatment or prevention of a proliferative disorder.

### Embodiment 201

A pharmaceutical formulation according to embodiment 200, wherein said proliferative disorder is a cancer.

### Embodiment 202

A pharmaceutical formulation according to embodiment 201, wherein said cancer is selected from an estrogen dependent cancer, breast cancer, prostate cancer, lung cancer, colorectal cancer, head and neck cancer, ovarian cancer, cervical cancer, bladder cancer, pancreatic cancer, gastrointestinal cancer, leukaemia, skin cancer, and lymphoma.

### Embodiment 203

A pharmaceutical formulation according to embodiment 202, wherein said cancer is breast, prostate, colorectal, head and neck or lung cancer.

### Embodiment 204

A pharmaceutical formulation according to embodiment 203, wherein said cancer is breast cancer.

### Embodiment 205

Use of a peptide according to any of embodiments 1 to 180 for therapy.

### Embodiment 206

Use of a peptide according to any of embodiments 1 to 180 in the treatment or prevention of a proliferative disorder.

### Embodiment 207

Use of a peptide according to any of embodiments 1 to 180 for the preparation of a pharmaceutical composition for the treatment or prevention of a proliferative disorder.

### Embodiment 208

Use according to embodiment 206 or embodiment 207, wherein said proliferative disorder is a cancer.

### Embodiment 209

Use according to embodiment 208, wherein said cancer is selected from an estrogen dependent cancer, breast cancer, prostate cancer, lung cancer, colorectal cancer, head and neck cancer, ovarian cancer, cervical cancer, bladder cancer, pancreatic cancer, gastrointestinal cancer, leukaemia, skin cancer, and lymphoma.

### Embodiment 210

A use according to embodiment 209, wherein said cancer is breast, prostate, colorectal, head and neck or lung cancer.

### Embodiment 211

Use according to embodiment 210, wherein said cancer is breast cancer.

### Embodiment 212

A method of treatment or prevention of a proliferative disorder, which comprises administration of an effective amount of a peptide according to any of embodiments 1 to 180 or a pharmaceutical formulation according to any of embodiments 199 to 204 to a patient in need thereof.

### Embodiment 213

A method according to embodiment 212, wherein said proliferative disorder is a cancer.

### Embodiment 214

A method according to embodiment 213, wherein said cancer is selected from an estrogen dependent cancer, breast cancer, prostate cancer, lung cancer, colorectal cancer, head and neck cancer, ovarian cancer, cervical cancer, bladder cancer, pancreatic cancer, gastrointestinal cancer, leukaemia, skin cancer, and lymphoma.

### Embodiment 215

A method according to embodiment 214, wherein said cancer is breast, prostate, colorectal, head and neck or lung cancer.

### Embodiment 216

A method according to embodiment 215, wherein said cancer is breast cancer.

All references, including publications, patent applications, and patents, cited herein are hereby incorporated by reference in their entirety and to the same extent as if each reference were individually and specifically indicated to be incorporated by reference and were set forth in its entirety herein (to the maximum extent permitted by law), regardless of any separately provided incorporation of particular documents made elsewhere herein.

The use of the terms “a” and “an” and “the” and similar referents in the context of describing the invention are to be construed to cover both the singular and the plural, unless otherwise indicated herein or clearly contradicted by context. For example, the phrase “the compound” is to be understood as referring to various “compounds” of the invention or particular described aspect, unless otherwise indicated.

Unless otherwise indicated, all exact values provided herein are representative of corresponding approximate values (e.g., all exact exemplary values provided with respect to a particular factor or measurement can be considered to also provide a corresponding approximate measurement, modified by “about,” where appropriate).

The description herein of any aspect or aspect of the invention using terms such as “comprising”, “having,” “including,” or “containing” with reference to an element or elements is intended to provide support for a similar aspect or aspect of the invention that “consists of”, “consists essentially of”, or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

### EXAMPLES

### Example 1

### Identification of BS1 in PRL

The prolactin molecule used in this example is a variant of PRL, wherein amino acid residues 1-11 has been deleted and which contains the mutations Q12S and G129R. The G129R mutation and 1-11 deletion disrupts BS2 binding, whereas the Q12S mutation has been introduced in order to ensure optimal activity of Methionine aminopeptidase (MetAP) leading to a more homogeneous product. MetAP are the enzymes responsible for the removal of the initiator NH₂-terminal methionine from newly synthesized proteins. For the purpose of this specification, this variant will be named vPRL.

The pET32-a(+) expression vector (Novagen, Madison Wis.) was used for expression of all proteins. Recombinant hPRL and mutated vPRL were produced in Escherichia coli as inclusion bodies. Solubilization in 8M Urea, 0.1 M Tris, 2-20 mM DTT, pH 8.5 buffer and following refolding by dilution into a 20 mM Tris, 0.05% Tween 20, pH 8.0. Protein purification was performed using Source30Q ion exchange columns (Amersham Biosciences) followed by a macro-prep Caramic Hydroxyapatite column (BioRad) and a final size-exclusion chromatography on a Sephadex G25 column.

ECD-PRL-R was refolded in two dilution steps, first in 0.4M Arginine, pH 8.5 and then diluted further in 20 mM Tris, 0.05% Tween 20, pH 8.0.

Amide hydrogen/deuterium exchange (HX) was initiated by a 10-fold dilution of vPRL in the presence or absence of ECD-PRL-R into a deuterated buffer (i.e. 20 mM Tris, 150 mM NaCl, 99% D₂O, pH 7.4 (uncorrected value)). Non-deuterated controls were prepared by dilution into an identical protiated buffer. All HX reactions were carried out at 30° C. and contained 6 μM vPRL variant in the absence or presence of 12 μM ECD-PRL-R. At appropriate time intervals, aliquots of the HX reaction were quenched by addition of an equal volume of ice-cold quenching buffer (1.25 M Tris(2-carboxyethyl)phosphine hydrochloride, adjusted to pH 2.0 using NaOH) resulting in a final pH of 2.5 (uncorrected value). Quenched samples were immediately frozen in liquid N₂ and stored at −80° C.

The samples were run on a cooled high pressure liquid chromatography-mass spectrometry system for rapid desalting and mass analysis as described (Rand et al. J. Biol. Chem. 281, 23018 (2006)). An example of raw data from two different peptides of vPRL are shown in FIG. 2.

Peptic peptides were identified in separate experiments using standard MS/MS methods. Average masses of peptide isotopic envelopes were determined from lockmass-corrected centroided data (processed using MassLynx software, Waters Inc.) using an Excel spreadsheet. Complete deuteration of control samples was achieved by incubation for 6 hrs at 90° C. Average back-exchange (i.e. deuterium loss) was measured to be approx. 15-20% for the analyzed peptides.

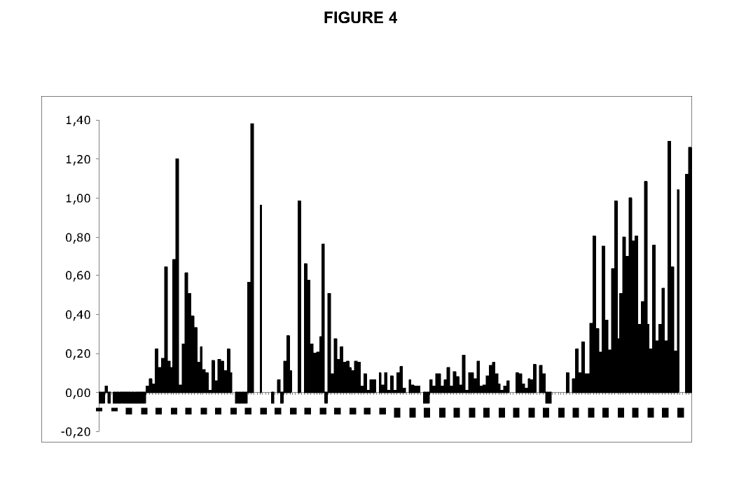

The HX time-course of 27 peptides, covering 90% of the primary sequence of vPRL, were monitored in the presence and absence ECD-PRL-R (FIGS. 2 and 3). vPRL peptides displaying reduced deuterium incorporation (>0.3 Da) after 1000 s HX in the presence of ECD-PRL-R were mapped onto the NMR structure of PRL and found to form a localized surface patch defined as BS1 (FIG. 4).

Consequently, BS1 is said to comprise the segments of PRL consisting of amino acid residues 20-36, 40-63, 66-83, 173-185 and 189-199.

### Example 2

### Mapping of the Receptor Binding Site 1 in Prolactin Using NMR Spectroscopy

### Protein Expression and Purification

Isotopically labeled PRL-G129R ([²H, ¹⁵N]PRL-G129R and [²H, ¹³C, ¹⁵N]PRL-G129R) for NMR studies were prepared by fermenting as for the corresponding unlabeled protein using a growth media containing ²H₂O and appropriately labeled nitrogen and carbon sources (¹⁵N-ammonium and ²H₇-D-glucose/¹³C₆-D-glucose).

### Preparation of NMR Samples

Samples of [²H, ¹⁵N]PRL-G129R and [²H, ¹³C, ¹⁵N]PRL-G129R at concentrations ranging between 0.2-0.5 mM were prepared in 2 mM NH₄HCO₃, 1 mM NaN₃ and 10% (v/v) ²H₂O (pH 8.0) (denoted NMR buffer). The complex between [²H, ¹⁵N]PRL-G129R (or [²H, ¹³C, ¹⁵N]PRL-G129R) and ECD-PRL-R was prepared by mixing [²H, ¹⁵N]PRL-G129R and ECD-PRL-R in a ratio 1:1.2. The binary complex was purified by gel filtration in 2 mM NH₄HCO₃ and 50 mM NaCl (pH 8.0) using a Superdex 75 Prep 26/60 column. Finally the complex was concentrated and exchanged into the NMR buffer. For assignment experiments and for the chemical shift perturbation measurements the buffer contained 10% ²H₂O. For the cross-saturation experiments the buffer contained 90-95% ²H₂O in order to quench potential amide proton mediated spin diffusion in [2H, ¹⁵N]PRL-G129R.

### NMR Experiments

NMR experiments were recorded at 35° C. on a Varian Inova 800 MHz instrument equipped with a triple-resonance cold probe, or a Bruker Avance 600 MHz spectrometer equipped with a room temperature triple-resonance probe. 2D-¹H, ¹⁵N-HSQC, 2D-¹H, ¹⁵N-TROSY, ¹⁵N-edited 3D-NOESY-HSQC, HNCO, HNCA, NHCOCA, HNCACB and HNCOCACB spectra were recorded using standard Bruker or Varian pulse sequences. The spectra were processed by the Felix software (Accelrys Software Inc.), peak picked by the Sparky software (T. D. Goddard and D. G. Kneller, SPARKY 3, University of California, San Francisco). Back-bone assignment was assisted by the AutoAssign software (D. E. Zimmerman, H. N. B. Moseley, C. A. Kulikowski, G. T. Montelione and Rutgers, The State University of New Jersey).

Cross-saturation experiments were recorded essentially as described by (Takahashi, H. et al., Nature Structural Biology 7, 220-223 (2000)). Thus, saturation of aliphatic resonances was achieved by trains of soft WURST-shaped inversion pulses with a length of 15 ms applied at 0.9 ppm. Reference spectra were recorded identically except for the offset for the saturation that was shifted to −5 ppm. Spectra were recorded in an interleaved fashion.

### Assignment of Back Bone Amide Groups in PRL and PRL-G129R

Chemical shift assignments for human PRL at pH 8.0 and 37° C. have been deposited in BioMagResBank with entry number 6643 by Teilum et al. J. Mol. Biol. 351, 810-823 (2005). 137 out of 182 main chain amide groups were assigned.

¹H, ¹⁵N-HSQC spectra of [²H, ¹⁵N]PRL-G129R were acquired at different temperatures and at 35° C. the ¹H, ¹⁵N-HSQC spectrum of [²H, ¹⁵N]PRL-G129R closely resembled the spectrum of ¹⁵N-PRL published by Teilum 2005. Some back bone amide signals shift their position in the spectrum of PRL-G129R relative to that of PRL, however major shifts can be directly attributed to the G129R mutation. Back-bone assignments were confirmed by standard assignment experiments, including HNCO, HNCACO, HNCA, HNCOCA, NHCACB and HNCOCACB, and a ¹⁵N-edited 3D-NOESY-HSQC spectrum of [²H, ¹⁵N]PRL-G129R served to further validate assignments using the sequential NOEs observed between back-bone amide groups (Table 1).

Back-Bone Assignment of [²H, ¹⁵N]PRL-G129R in Complex with ECD-PRL-R

Back-bone amide resonances in PRL-G129R in complex with ECD-PRL-R were assigned using a set of TROSY based 3D triple resonance experiments, including HNCO, HNCA, NHCOCA, HNCACB and HNCOCACB, supplemented by a 3D ¹⁵N-edited NOESY-HSQC experiment. The back-bone assignments are shown in Table 1.

### Mapping of Binding Interface Using the Chemical Shift Perturbation Method

A ¹H, ¹⁵N-TROSY spectrum was recorded for the one-to-one complex between [²H, ¹⁵N]PRL-G129R and ECD-PRL-R at 800 MHz. Only signals corresponding to the PRL part of the complex is observed as ECD-PRL-R is unlabeled. A reference spectrum was acquired for [²H, ¹⁵N]PRL-G129R under identical conditions.

Chemical shift differences for back-bone amide protons in PRL-G129R in the free and receptor bound states were calculated from the back-bone assignments for free PRL-G129R and PRL-G129R bound to ECD-PRL-R (Table 1). The observed chemical shift difference for each amino acid residue induced by PRL binding to ECD-PRL-R is shown graphically in FIG. 4.

TABLE 1
Back-bone chemical shift (δ) assignments of amide proton (H), nitrogen (N), carbonyl carbon
(CO), α-carbon (Cα) and β-carbon (Cβ) for free PRL-G129R and for PRL-G129R in complex
with ECD-PRL-R. Listed are chemical shift differences (δ_{bound} − δ_{free}) for amide proton and
nitrogen (Δδ_{H} and Δδ_{N}, respectively), from which the combined proton-nitrogen chemical shift
difference index (ΔCS) is calculated as ΔCS = [(Δδ_{H})² + (0.1 × Δδ_{N})²]^{0.5}.

| | | | | | | PRL-G129R/ECD-PRL-R | | | | | | | |
|---|---|---|---|---|---|---|---|---|---|---|---|---|---|
| | Free PRL-G129R | | | | | Complex | | | | | Comparison | | |
| Residue | δ_{H} | δ_{N} | δ_{CO}a | δ_{Cα} | δ_{Cβ} | δ_{H} | δ_{N} | δ_{CO}a | δ_{Cα} | δ_{Cβ} | Δδ_{H} | Δδ_{N} | ΔCS^{b} |
| L1 | | | | | | | | | | | | | (###) |
| P2 | | | | | | | | | | | | | |
| I3 | 8.07 | 121.8 | 176.6 | 61.0 | 38.0 | 8.08 | 121.9 | 176.6 | 61.0 | 38.0 | 0.01 | 0.10 | 0.03 |
| C4 | | | | | | | | | | | | | (###) |
| P5 | | | | | | | | | | | | | (###) |
| G6 | | | | | | | | | | | | | (###) |
| G7 | | | | | | | | | | | | | (###) |
| A8 | | | | | | | | | | | | | (###) |
| A9 | | | | | | | | | | | | | (###) |
| R10 | | | | | | | | | | | | | (###) |
| C11 | | | | | | | | | | | | | (###) |
| Q12 | | | | | | | | | | | | | (###) |
| V13^{c} | 7.95 | 122.4 | 176.1 | 62.3 | 32.2 | | | | | | | | (###) |
| T14^{c} | 8.31 | 118.0 | 176.4 | 61.8 | 70.3 | | | | | | | | (###) |
| L15 | | | | | | | | | | | | | (###) |
| R16 | | | | | | | | | | | | | (###) |
| D17 | 7.65 | 120.7 | 177.7 | 57.2 | 40.5 | 7.65 | 120.8 | 177.6 | 57.1 | 40.7 | 0.00 | 0.10 | 0.03 |
| L18 | 8.11 | 121.2 | 179.2 | 57.8 | 41.5 | 8.17 | 121.3 | 179.5 | 57.9 | 41.2 | 0.06 | 0.10 | 0.07 |
| F19 | 8.49 | 119.5 | 179.8 | 62.7 | 38.9 | 8.52 | 119.6 | 179.6 | 62.9 | 38.7 | 0.03 | 0.10 | 0.04 |
| D20 | 8.56 | 121.5 | 178.2 | 57.5 | 39.7 | 8.60 | 120.8 | 178.4 | 57.4 | 39.8 | 0.04 | −0.70 | 0.22 |
| R21 | 7.77 | 118.5 | 179.2 | 57.6 | 28.9 | 7.76 | 118.1 | 179.1 | 57.3 | | −0.01 | −0.40 | 0.13 |
| A22 | 8.19 | 124.1 | 178.8 | 55.2 | 17.4 | 8.34 | 123.8 | 178.8 | 55.5 | 17.1 | 0.15 | −0.30 | 0.18 |
| V23 | 8.11 | 116.4 | 178.3 | 64.1 | 30.8 | 7.87 | 114.5 | 177.9 | 63.3 | | −0.24 | −1.90 | 0.65 |
| V24 | 7.42 | 123.3 | 179.1 | 66.2 | 31.0 | 7.43 | 123.8 | 179.2 | 66.5 | 31.0 | 0.01 | 0.50 | 0.16 |
| L25 | 7.64 | 121.6 | 179.2 | 57.4 | 42.2 | 7.67 | 121.2 | 179.4 | 57.3 | | 0.03 | −0.40 | 0.13 |
| S26 | 9.23 | 118.0 | 178.9 | 62.3 | 61.5 | 9.65 | 119.7 | 178.7 | 62.3 | 61.0 | 0.42 | 1.70 | 0.68 |
| H27 | 8.14 | 127.4 | 178.1 | 59.5 | 29.4 | 8.12 | 131.2 | 178.4 | 61.1 | | −0.02 | 3.80 | 1.20 |
| Y28 | 7.88 | 122.6 | 177.4 | 59.7 | 38.2 | 7.84 | 122.6 | 177.5 | 60.0 | 39.5 | −0.04 | 0.00 | 0.04 |
| I29 | 8.83 | 120.5 | 178.0 | 65.7 | 37.8 | 9.07 | 120.3 | 181.1 | 65.7 | 37.1 | 0.24 | −0.20 | 0.25 |
| H30 | 8.21 | 122.2 | 178.4 | 58.5 | 27.7 | 7.98 | 120.4 | 178.8 | 59.1 | 26.2 | −0.23 | −1.80 | 0.61 |
| N31 | 8.13 | 122.7 | 177.9 | 56.6 | 37.5 | 8.08 | 121.1 | 177.6 | 57.1 | 38.2 | −0.05 | −1.60 | 0.51 |
| L32 | 8.54 | 122.2 | 177.6 | 57.5 | 42.3 | 8.36 | 121.1 | 177.4 | 57.3 | 42.2 | −0.18 | −1.10 | 0.39 |
| S33 | 8.58 | 117.7 | 179.6 | 62.1 | 62.5 | 8.27 | 118.1 | 179.4 | 62.2 | | −0.31 | 0.40 | 0.33 |
| S34 | 8.05 | 122.6 | 177.6 | 62.0 | 62.6 | 8.17 | 122.3 | 177.6 | 61.8 | | 0.12 | −0.30 | 0.15 |
| E35 | 8.29 | 124.9 | 176.8 | 58.9 | 29.0 | 8.12 | 124.4 | 176.7 | 58.9 | 28.9 | −0.17 | −0.50 | 0.23 |
| M36 | 8.82 | 121.0 | 179.2 | 59.6 | 32.8 | 8.72 | 121.2 | 179.1 | 59.7 | 32.4 | −0.10 | 0.20 | 0.12 |
| F37 | 7.98 | 119.4 | 177.4 | 61.8 | 38.4 | 8.08 | 119.4 | 177.2 | 61.9 | 38.1 | 0.10 | 0.00 | 0.10 |
| S38 | 8.21 | 115.9 | 176.5 | 61.7 | 62.8 | 8.22 | 115.9 | 176.2 | 61.7 | 62.7 | 0.01 | 0.00 | 0.01 |
| E39 | 8.38 | 122.5 | 177.2 | 58.5 | 28.7 | 8.23 | 122.3 | 177.1 | 58.5 | 28.6 | −0.15 | −0.20 | 0.16 |
| F40 | 8.35 | 122.8 | 178.8 | 61.8 | 39.8 | 8.30 | 122.7 | 178.7 | 61.8 | 40.0 | −0.05 | −0.10 | 0.06 |
| D41 | 8.68 | 121.3 | 178.1 | 55.6 | 41.7 | 8.74 | 121.8 | 176.8 | 55.2 | 42.1 | 0.06 | 0.50 | 0.17 |
| K42 | 8.04 | 118.6 | 178.8 | 58.7 | 31.5 | 8.03 | 118.1 | 178.8 | 58.7 | 31.6 | −0.01 | −0.50 | 0.16 |
| R43 | 7.32 | 117.7 | 178.1 | 56.9 | 29.9 | 7.21 | 117.7 | 178.0 | 56.9 | 29.5 | −0.11 | 0.00 | 0.11 |
| Y44 | 8.10 | 116.1 | 177.5 | 59.5 | 40.3 | 8.11 | 115.4 | 177.6 | 59.4 | | 0.01 | −0.70 | 0.22 |
| T45 | 7.67 | 110.3 | 176.6 | 62.7 | 69.0 | 7.70 | 110.6 | 174.9 | 61.7 | 67.9 | 0.03 | 0.30 | 0.10 |
| H46 | 8.50 | 125.4 | 175.8 | 57.8 | 28.6 | | | | | | | | ($$$) |
| G47 | | | | | | | | | | | | | (###) |
| R48 | | | | | | | | | | | | | (###) |
| G49 | | | | | | | | | | | | | (###) |
| F50 | | | | | | | | | | | | | (###) |
| I51 | 7.56 | 122.8 | 176.4 | 61.1 | 37.4 | 8.11 | 122.4 | 178.2 | 62.9 | ca.37 | 0.55 | −0.40 | 0.56 |
| T52 | 7.75 | 117.0 | 176.5 | 62.3 | 68.8 | 7.02 | 113.3 | 176.2 | 64.0 | 68.6 | −0.73 | −3.70 | 1.38 |
| K53 | | | | | | 7.07 | 120.4 | 176.1 | 55.5 | 31.8 | | | (¤¤¤) |
| A54 | | | | | | 7.32 | 124.0 | 176.2 | 51.7 | 17.4 | | | (¤¤¤) |
| I55 | 7.89 | 120.6 | 177.7 | 60.3 | 37.9 | 6.94 | 120.1 | 179.2 | 60.1 | 36.7 | −0.95 | −0.50 | 0.96 |
| N56 | | | | | | 10.36 | 124.1 | 176.6 | 54.5 | 37.1 | | | (¤¤¤) |
| S57 | | | | | | 7.95 | 113.5 | 176.1 | 59.8 | 65.2 | | | (¤¤¤) |
| C58 | | | | | | 8.42 | 123.6 | | 53.0 | 34.2 | | | (¤¤¤) |
| H59 | | | | | | | | | | | | | (###) |
| T60 | 7.40 | 110.0 | 176.4 | 61.6 | 67.9 | | | | | | | | ($$$) |
| S61 | 7.88 | 119.8 | 176.9 | 58.0 | | 7.86 | 119.6 | 176.5 | 61.1 | 64.1 | −0.02 | −0.20 | 0.07 |
| S62 | | | | | | | | | | | | | (###) |
| L63 | 7.50 | 124.6 | 174.9 | 54.0 | 41.6 | 7.49 | 125.1 | 175.0 | 53.8 | 42.2 | −0.01 | 0.50 | 0.16 |
| A64 | 8.32 | 128.3 | 176.3 | 51.5 | 17.0 | 8.39 | 129.2 | 176.0 | 51.4 | 16.6 | 0.07 | 0.90 | 0.29 |
| T65 | 8.00 | 114.7 | 176.9 | 57.7 | 68.4 | 8.09 | 114.9 | 177.0 | 57.4 | 68.0 | 0.09 | 0.20 | 0.11 |
| P66 | | | | | | | | | | | | | |
| E67 | | | | | | 9.04 | 125.4 | 179.0 | 55.7 | 30.8 | | | (¤¤¤) |
| D68 | 7.61 | 118.6 | 175.9 | 52.6 | 42.2 | 7.63 | 115.5 | 173.8 | 52.4 | 42.7 | 0.02 | −3.10 | 0.98 |
| K69 | | | | | | 8.16 | 121.9 | 175.3 | 58.7 | 30.9 | | | (¤¤¤) |
| E70 | 8.32 | 122.3 | 177.9 | 58.9 | 28.2 | 7.76 | 121.2 | 177.9 | 59.0 | 27.8 | −0.56 | −1.10 | 0.66 |
| Q71 | 8.48 | 120.3 | 179.2 | 57.8 | 28.1 | 9.01 | 121.0 | 179.9 | 58.3 | 28.0 | 0.53 | 0.70 | 0.57 |
| A72 | 7.92 | 123.3 | 179.2 | 54.1 | 17.6 | 8.03 | 122.6 | 180.1 | 54.5 | 17.5 | 0.11 | −0.70 | 0.25 |
| Q73 | 8.02 | 117.4 | 178.8 | 57.3 | 28.0 | 8.22 | 117.4 | 178.9 | 57.8 | 28.0 | 0.20 | 0.00 | 0.20 |
| Q74 | 7.37 | 116.9 | 176.9 | 55.3 | 28.4 | 7.29 | 116.3 | 177.4 | 55.5 | 28.5 | −0.08 | −0.60 | 0.21 |
| M75 | 7.26 | 121.9 | 176.4 | 55.5 | 32.6 | 7.02 | 122.4 | 176.1 | 55.3 | 31.7 | −0.24 | 0.50 | 0.29 |
| N76 | 8.96 | 125.5 | 175.9 | 53.1 | 38.9 | 8.96 | 127.9 | 176.0 | 53.6 | 38.9 | 0.00 | 2.40 | 0.76 |
| E77 | | | | | | | | | | | | | (###) |
| K78 | 8.17 | 121.9 | 176.9 | 59.5 | 30.7 | 8.20 | 123.5 | 176.2 | 59.8 | 30.9 | 0.03 | 1.60 | 0.51 |
| D79 | 7.88 | 121.7 | 178.6 | 56.4 | 39.7 | 7.90 | 121.4 | 178.7 | 60.0 | 39.6 | 0.02 | −0.30 | 0.10 |
| F80 | 8.29 | 123.3 | 178.9 | 59.2 | 39.0 | 8.45 | 124.0 | 178.9 | 58.8 | | 0.16 | 0.70 | 0.27 |
| L81 | 8.29 | 119.9 | 178.8 | 528.2 | 40.9 | 8.35 | 119.4 | 179.2 | 58.2 | 40.4 | 0.06 | −0.50 | 0.17 |
| S82 | 8.10 | 114.8 | 178.5 | 61.6 | 62.7 | 7.87 | 114.9 | 178.7 | 61.6 | | −0.23 | 0.10 | 0.23 |
| L83 | 7.94 | 125.4 | 176.9 | 57.8 | 41.3 | 8.06 | 125.1 | 177.0 | 57.7 | 41.3 | 0.12 | −0.30 | 0.15 |
| I84 | 7.85 | 120.0 | 177.7 | 65.7 | 37.8 | 7.95 | 120.4 | 178.5 | 65.8 | 37.8 | 0.10 | 0.40 | 0.16 |
| V85 | 7.83 | 119.2 | 177.6 | 67.2 | 30.8 | 7.81 | 119.6 | 177.6 | 67.4 | 30.4 | −0.02 | 0.40 | 0.13 |
| S86 | 8.47 | 117.7 | | 62.6 | 63.5 | 8.36 | 117.7 | 177.3 | 62.6 | 62.3 | −0.11 | 0.00 | 0.11 |
| I87 | 8.41 | 123.2 | 176.8 | 66.0 | 37.6 | 8.38 | 122.7 | 176.6 | 65.9 | 37.7 | −0.03 | −0.50 | 0.16 |
| L88 | 8.10 | 120.9 | 179.0 | 58.4 | 42.3 | 8.19 | 121.3 | 179.4 | 58.2 | 42.1 | 0.09 | 0.40 | 0.16 |
| R89 | 8.94 | 119.7 | 179.9 | 59.0 | 28.9 | 8.95 | 119.8 | 179.8 | 58.9 | 28.9 | 0.01 | 0.10 | 0.03 |
| S90 | 7.88 | 116.0 | 179.7 | 60.6 | 63.1 | 7.87 | 116.3 | 179.8 | 60.5 | 63.1 | −0.01 | 0.30 | 0.10 |
| W91 | 7.72 | 123.3 | 175.7 | 59.1 | 29.5 | 7.73 | 123.3 | 175.5 | 59.1 | 29.3 | 0.01 | 0.00 | 0.01 |
| N92 | 7.31 | 121.4 | 176.5 | 57.4 | 38.4 | 7.30 | 121.2 | 176.6 | 57.4 | 38.3 | −0.01 | −0.20 | 0.06 |
| E93 | 9.18 | 121.0 | 177.2 | 60.6 | 26.5 | 9.17 | 121.2 | 177.2 | 60.7 | 26.4 | −0.01 | 0.20 | 0.06 |
| P94 | | | | | | | | | | | | | |
| L95 | 7.78 | 115.4 | 178.3 | 57.8 | 40.6 | 7.68 | 115.4 | 178.2 | 57.9 | 41.1 | −0.10 | 0.00 | 0.10 |
| Y96 | 7.80 | 121.9 | 178.3 | 60.8 | 37.2 | 7.78 | 121.8 | 178.2 | 60.7 | 36.9 | −0.02 | −0.10 | 0.04 |
| H97 | 8.16 | 121.1 | 179.3 | 59.5 | | 8.19 | 121.4 | 179.3 | 59.8 | | 0.03 | 0.30 | 0.10 |
| L98 | 8.80 | 123.6 | 176.9 | 58.3 | 41.0 | 8.79 | 123.6 | 176.9 | 58.3 | 40.9 | −0.01 | 0.00 | 0.01 |
| V99 | 7.73 | 118.1 | 177.6 | 66.9 | 31.1 | 7.67 | 117.9 | 177.6 | 66.9 | 31.0 | −0.06 | −0.20 | 0.09 |
| T100 | 7.57 | 115.1 | 177.4 | 66.4 | 68.4 | 7.58 | 115.1 | 177.3 | 66.4 | 68.4 | 0.01 | 0.00 | 0.01 |
| E101 | 8.84 | 122.9 | 177.6 | 58.1 | 28.8 | 8.92 | 122.7 | 177.7 | 58.2 | 28.7 | 0.08 | −0.20 | 0.10 |
| V102 | 8.47 | 121.7 | 180.4 | 65.6 | 30.1 | 8.51 | 122.1 | 180.5 | 65.7 | 29.6 | 0.04 | 0.40 | 0.13 |
| R103 | 8.19 | 120.7 | 178.6 | 58.9 | 29.4 | 8.17 | 120.7 | 178.6 | 58.9 | 29.4 | −0.02 | 0.00 | 0.02 |
| G104 | 7.42 | 105.3 | 177.7 | 44.5 | | 7.42 | 105.3 | 177.7 | 44.5 | | 0.00 | 0.00 | 0.00 |
| M105 | 7.48 | 123.3 | 174.4 | 55.8 | 32.6 | 7.49 | 123.5 | 174.4 | 55.9 | 32.5 | 0.01 | 0.20 | 0.06 |
| Q106 | 8.60 | 125.3 | 176.4 | 57.5 | 27.9 | 8.58 | 125.4 | 176.4 | 57.5 | 27.8 | −0.02 | 0.10 | 0.04 |
| E107 | 8.66 | 120.7 | 176.8 | 55.6 | 28.0 | 8.66 | 120.8 | 176.8 | 55.6 | 27.9 | 0.00 | 0.10 | 0.03 |
| A108 | 7.77 | 126.1 | 175.7 | 50.5 | 17.7 | 7.76 | 126.0 | 175.6 | 50.5 | 17.6 | −0.01 | −0.10 | 0.03 |
| P109 | | | | | | | | | | | | | |
| E110 | | | | | | | | | | | | | (###) |
| A111 | | | | | | | | | | | | | (###) |
| I112 | 7.25 | 116.0 | 179.7 | 63.6 | 37.1 | 7.26 | 115.8 | 179.6 | 63.7 | 36.7 | 0.01 | −0.20 | 0.06 |
| L113 | 7.83 | 122.2 | 176.5 | 58.1 | 40.5 | 7.84 | 122.1 | 176.4 | 58.1 | 40.3 | 0.01 | −0.10 | 0.03 |
| S114 | 8.48 | 113.1 | 177.8 | 61.2 | 62.4 | 8.47 | 112.8 | 177.7 | 61.1 | 62.6 | −0.01 | −0.30 | 0.10 |
| K115 | 7.19 | 122.3 | 177.8 | 59.0 | 32.1 | 7.17 | 122.6 | 178.1 | 59.1 | | −0.02 | 0.30 | 0.10 |
| A116 | 8.29 | 124.9 | 177.1 | 55.3 | 16.8 | 8.30 | 125.0 | 177.0 | 55.4 | 16.7 | 0.01 | 0.10 | 0.03 |
| V117 | 8.24 | 118.7 | 178.8 | 65.5 | 31.6 | 8.25 | 118.5 | 178.8 | 65.5 | 31.6 | 0.01 | −0.20 | 0.06 |
| E118 | 7.56 | 122.3 | 179.1 | 58.6 | 29.3 | 7.45 | 122.5 | 179.0 | 58.6 | | −0.11 | 0.20 | 0.13 |
| I119 | 8.94 | 122.1 | 178.9 | 66.0 | 37.2 | 8.94 | 122.2 | 178.9 | 66.1 | 36.9 | 0.00 | 0.10 | 0.03 |
| E120 | 8.42 | 126.2 | 178.6 | 60.4 | 28.1 | 8.47 | 125.9 | 178.8 | 60.5 | 28.0 | 0.05 | −0.30 | 0.11 |
| E121 | 7.72 | 119.6 | 179.9 | 58.7 | 28.9 | 7.67 | 119.8 | 178.0 | 60.5 | 29.1 | −0.05 | 0.20 | 0.08 |
| Q122 | 9.03 | 117.9 | 179.6 | 57.4 | 26.9 | 9.05 | 117.8 | 179.6 | 57.4 | 26.8 | 0.02 | −0.10 | 0.04 |
| T123 | 8.51 | 118.3 | 179.2 | 67.5 | | 8.53 | 118.9 | 179.1 | 67.5 | | 0.02 | 0.60 | 0.19 |
| K124 | 7.17 | 123.2 | 176.9 | 59.9 | 31.1 | 7.18 | 123.2 | 176.8 | 60.0 | 31.2 | 0.01 | 0.00 | 0.01 |
| R125 | 7.79 | 120.8 | 179.9 | 58.3 | | 7.71 | 120.6 | 180.0 | 59.2 | 29.2 | −0.08 | −0.20 | 0.10 |
| L126 | 8.83 | 124.3 | 179.8 | 57.4 | 39.4 | 8.80 | 124.6 | 179.8 | 57.5 | 39.6 | −0.03 | 0.30 | 0.10 |
| L127 | 8.53 | 123.5 | 178.3 | 58.4 | 40.0 | 8.50 | 123.3 | 178.3 | 58.2 | | −0.03 | −0.20 | 0.07 |
| E128 | 7.85 | 119.5 | | 59.1 | 28.2 | 7.82 | 120.0 | 177.7 | 58.9 | 29.0 | −0.03 | 0.50 | 0.16 |
| R129 | 7.71 | 119.4 | | 58.5 | 29.0 | 7.70 | 119.3 | 179.9 | 58.6 | | −0.01 | −0.10 | 0.03 |
| M130 | 8.70 | 118.9 | 178.6 | 55.2 | 28.3 | 8.66 | 118.9 | 178.5 | 55.1 | 28.3 | −0.04 | 0.00 | 0.04 |
| E131 | 8.73 | 121.6 | 179.0 | 59.7 | 28.6 | 8.65 | 121.5 | 179.0 | 59.7 | 28.5 | −0.08 | −0.10 | 0.09 |
| L132 | 7.51 | 122.6 | 180.0 | 57.3 | 40.9 | 7.45 | 122.2 | 180.1 | 57.3 | 40.8 | −0.06 | −0.40 | 0.14 |
| I133 | 7.97 | 122.8 | 180.0 | 66.3 | 37.3 | 8.11 | 123.0 | 179.9 | 66.5 | 37.4 | 0.14 | 0.20 | 0.15 |
| V134 | 8.67 | 121.2 | | 67.3 | 30.8 | 8.69 | 120.9 | 178.1 | 67.4 | 31.0 | 0.02 | −0.30 | 0.10 |
| S135 | 7.77 | 114.4 | 177.7 | 61.1 | 63.1 | 7.74 | 114.5 | 177.7 | 61.1 | 63.0 | −0.03 | 0.10 | 0.04 |
| Q136 | 7.47 | 118.9 | 175.0 | 56.8 | 28.9 | 7.46 | 118.9 | 175.0 | 57.0 | 28.6 | −0.01 | 0.00 | 0.01 |
| V137 | 7.97 | 117.9 | 177.3 | 63.5 | 32.5 | 7.98 | 117.8 | 177.4 | 63.6 | 32.3 | 0.01 | −0.10 | 0.03 |
| H138 | 8.56 | 119.8 | 175.6 | 53.6 | 29.8 | 8.62 | 119.8 | 175.5 | 53.6 | 29.8 | 0.06 | 0.00 | 0.06 |
| P139 | | | | | | | | | | | | | |
| E140 | 9.11 | 121.1 | 177.5 | 56.6 | 28.2 | | | | | | | | ($$$) |
| T141 | 7.94 | 118.9 | 176.7 | 62.7 | 69.4 | 7.91 | 119.2 | 176.6 | 62.8 | 69.3 | −0.03 | 0.30 | 0.10 |
| K142 | 8.29 | 127.0 | 174.3 | 55.6 | 32.4 | 8.30 | 127.3 | 174.3 | 55.5 | 32.4 | 0.01 | 0.30 | 0.10 |
| E143 | 8.39 | 123.4 | 176.4 | 56.2 | 29.5 | 8.42 | 123.5 | 176.4 | 56.2 | 29.5 | 0.03 | 0.10 | 0.04 |
| N144 | 8.36 | 121.1 | 176.2 | 52.9 | 38.4 | 8.38 | 121.1 | | 52.9 | 38.3 | 0.02 | 0.00 | 0.02 |
| E145 | 8.31 | 123.7 | 175.0 | 56.1 | 29.7 | 8.37 | 123.8 | 175.0 | 56.1 | 29.6 | 0.06 | 0.10 | 0.07 |
| I146 | 7.99 | 123.4 | 176.1 | 60.4 | 38.0 | 7.99 | 123.2 | 176.1 | 60.4 | 37.9 | 0.00 | −0.20 | 0.06 |
| Y147 | 7.44 | 124.6 | 175.0 | 54.3 | 37.6 | 7.37 | 124.2 | 174.9 | 54.1 | 37.7 | −0.07 | −0.40 | 0.14 |
| P148 | | | | | | | | | | | | | |
| V149 | 8.14 | 124.0 | 176.0 | 61.4 | 32.6 | 8.19 | 124.4 | 175.9 | 61.5 | 32.4 | 0.05 | 0.40 | 0.14 |
| W150 | 8.78 | 129.6 | 176.1 | 56.8 | 28.1 | 8.78 | 129.9 | 176.0 | 56.9 | 27.8 | 0.00 | 0.30 | 0.09 |
| S151 | | | | | | | | | | | | | (###) |
| G152 | | | | | | | | | | | | | (###) |
| L153^{c} | | | | | | 7.37 | 124.9 | 174.0 | 58.0 | | | | (###) |
| P154 | | | | | | | | | | | | | |
| S155 | 7.47 | 114.6 | 179.1 | 60.4 | 62.7 | | | | | | | | ($$$) |
| L156 | 7.58 | 121.2 | 175.1 | 56.0 | 40.8 | | | | | | | | ($$$) |
| Q157 | | | | | | 7.29 | 115.6 | 177.8 | 54.7 | 27.7 | | | (¤¤¤) |
| M158 | 7.10 | 121.0 | 176.1 | 55.5 | 32.6 | 7.07 | 121.3 | 176.2 | 55.8 | 32.6 | −0.03 | 0.30 | 0.10 |
| A159 | 8.22 | 125.2 | 175.8 | 53.2 | 18.5 | | | | | | | | ($$$) |
| D160 | 7.82 | 119.2 | 178.2 | 53.4 | 41.8 | 7.79 | 119.4 | 178.4 | 53.6 | 42.0 | −0.03 | 0.20 | 0.07 |
| E161 | 8.74 | 128.0 | 175.7 | 60.0 | 29.5 | 8.74 | 128.7 | 175.7 | 60.1 | 29.3 | 0.00 | 0.70 | 0.22 |
| E162 | 8.54 | 120.0 | 177.8 | 59.9 | 28.3 | 8.58 | 120.3 | 177.7 | 60.1 | 28.7 | 0.04 | 0.30 | 0.10 |
| S163 | 8.07 | 118.0 | 179.5 | 61.7 | 62.7 | 8.01 | 117.2 | 179.2 | 61.8 | 62.8 | −0.06 | −0.80 | 0.26 |
| R164 | 8.32 | 126.1 | 177.0 | 59.5 | 29.8 | 8.33 | 126.4 | 177.0 | 59.3 | 29.8 | 0.01 | 0.30 | 0.10 |
| L165 | 8.77 | 119.6 | 178.4 | 58.1 | 40.9 | 8.77 | 119.3 | 178.3 | 57.8 | 41.1 | 0.00 | −0.30 | 0.09 |
| S166 | 8.02 | 115.6 | 180.0 | 61.5 | 62.7 | 7.81 | 114.7 | 179.3 | 59.8 | 62.8 | −0.21 | −0.90 | 0.35 |
| A167 | 7.88 | 127.0 | 176.3 | 54.9 | 17.8 | 7.73 | 124.5 | 175.8 | 54.9 | 17.8 | −0.15 | −2.50 | 0.80 |
| Y168 | 8.46 | 121.7 | | 62.4 | 39.1 | 8.30 | 120.8 | 181.9 | 62.3 | 39.3 | −0.16 | −0.90 | 0.33 |
| Y169 | 8.74 | 122.0 | 177.3 | 62.3 | 38.5 | 8.82 | 122.6 | 177.1 | 62.4 | 38.4 | 0.08 | 0.60 | 0.21 |
| N170 | 8.49 | 117.8 | 177.4 | 56.6 | 38.9 | 8.94 | 119.7 | 177.5 | 55.7 | 37.4 | 0.45 | 1.90 | 0.75 |
| L171 | 8.08 | 123.4 | 177.3 | 58.7 | 41.9 | 7.95 | 124.5 | 178.1 | 58.9 | 42.0 | −0.13 | 1.10 | 0.37 |
| L172 | 8.53 | 119.6 | 178.9 | 57.4 | 40.7 | 8.38 | 120.1 | 178.9 | 57.5 | 41.0 | −0.15 | 0.50 | 0.22 |
| H173 | 8.95 | 124.9 | 180.1 | 60.1 | 29.8 | 9.43 | 126.2 | 180.3 | 59.3 | 27.4 | 0.48 | 1.30 | 0.63 |
| C174 | 8.62 | 121.6 | 178.7 | 58.8 | 38.9 | 8.88 | 124.6 | 179.2 | 59.0 | | 0.26 | 3.00 | 0.98 |
| L175 | 8.69 | 125.8 | 175.9 | 57.3 | 39.8 | 8.80 | 126.6 | 175.6 | 56.9 | 39.9 | 0.11 | 0.80 | 0.28 |
| R176 | 7.88 | 123.9 | 179.2 | 59.1 | 28.8 | 8.06 | 125.4 | 179.8 | 59.9 | | 0.18 | 1.50 | 0.51 |
| R177 | 7.80 | 121.2 | 177.7 | 58.6 | 29.3 | 7.16 | 119.7 | 176.4 | 58.9 | 30.2 | −0.64 | −1.50 | 0.80 |
| D178 | 8.96 | 123.6 | 179.6 | 57.1 | | 8.99 | 121.4 | 177.3 | 56.8 | 37.9 | 0.03 | −2.20 | 0.70 |
| S179 | 8.64 | 117.8 | 178.6 | 61.8 | 62.5 | 8.46 | 114.7 | 179.0 | 61.1 | | −0.18 | −3.10 | 1.00 |
| H180 | 7.71 | 127.2 | 177.4 | 58.8 | 29.8 | 7.44 | 129.5 | 177.6 | 62.6 | 29.3 | −0.27 | 2.30 | 0.78 |
| K181 | 7.69 | 120.6 | 177.0 | 59.3 | 31.3 | 7.84 | 123.1 | 177.0 | 59.2 | 29.8 | 0.15 | 2.50 | 0.80 |
| I182 | 8.13 | 117.5 | 177.2 | 64.8 | 36.6 | 7.98 | 116.5 | 176.4 | 64.1 | 36.4 | −0.15 | −1.00 | 0.35 |
| D183 | 7.34 | 120.2 | 176.9 | 57.3 | 41.8 | 6.93 | 119.5 | 177.2 | 56.5 | 41.6 | −0.41 | −0.70 | 0.47 |
| N184 | 7.86 | 117.8 | 177.5 | 55.9 | 37.7 | 8.38 | 120.8 | | 55.0 | 39.3 | 0.52 | 3.00 | 1.08 |
| Y185 | 9.06 | 121.5 | 178.9 | 55.9 | 36.5 | 9.07 | 120.4 | | 56.0 | 36.0 | 0.01 | −1.10 | 0.35 |
| L186 | 8.86 | 122.6 | | 58.0 | 41.0 | 8.66 | 122.9 | 177.8 | 58.0 | 41.6 | −0.20 | 0.30 | 0.22 |
| K187 | 7.89 | 120.7 | | 60.3 | 31.5 | 8.19 | 122.9 | 179.0 | 60.4 | | 0.30 | 2.20 | 0.76 |
| L188 | 7.76 | 123.0 | 179.9 | 57.6 | 40.9 | 7.68 | 123.8 | 179.7 | 56.7 | 42.6 | −0.08 | 0.80 | 0.27 |
| L189 | 8.62 | 122.6 | 179.8 | 57.4 | 41.3 | 8.61 | 121.5 | 179.3 | 57.0 | 41.6 | −0.01 | −1.10 | 0.35 |
| K190 | 8.51 | 122.1 | 178.2 | 59.6 | 31.7 | 8.96 | 123.0 | 178.5 | 59.6 | 31.9 | 0.45 | 0.90 | 0.53 |
| C191 | 7.70 | 118.5 | 178.1 | 59.7 | 41.3 | 7.62 | 119.3 | 179.4 | 61.0 | 41.7 | −0.08 | 0.80 | 0.27 |
| R192 | 8.05 | 120.7 | 176.6 | 58.9 | 30.4 | 8.85 | 123.9 | 177.1 | 60.0 | 31.5 | 0.80 | 3.20 | 1.29 |
| I193 | 8.22 | 118.3 | 178.0 | 63.4 | 37.6 | 8.86 | 118.0 | 178.2 | 64.0 | 38.0 | 0.64 | −0.30 | 0.65 |
| I194 | 7.82 | 118.8 | 176.8 | 60.2 | 35.4 | 7.91 | 118.2 | 177.2 | 59.4 | 34.2 | 0.09 | −0.60 | 0.21 |
| H195 | 7.18 | 117.7 | 176.9 | 55.4 | 29.5 | 6.58 | 115.0 | 177.6 | 54.6 | | −0.60 | −2.70 | 1.04 |
| N196 | | | | | | 7.32 | 120.3 | 176.3 | 54.3 | 36.6 | | | (¤¤¤) |
| N197 | | | | | | 9.21 | 114.0 | 174.3 | 53.8 | 36.2 | | | (¤¤¤) |
| N198 | 7.99 | 120.8 | 174.5 | 52.7 | | 7.35 | 117.9 | 174.1 | 52.0 | 38.8 | −0.64 | −2.90 | 1.12 |
| C199 | 7.75 | 124.9 | 174.0 | 56.3 | 44.6 | 8.80 | 127.1 | 176.1 | 58.2 | 43.6 | 1.05 | 2.20 | 1.26 |

^{a}carbonyl chemical shift for preceding residue
^{b}Combined proton-nitrogen chemical shift difference index is calculated as ΔCS = [(Δδ_{H})² + (0.1 × Δδ_{N})²]^{0.5}. (###) mark residues where no back-bone amide assignment was obtained in neither the free nor the bound state, ($$$) and (¤¤¤) cells mark residues where assignment was obtained for the free or bound state only, respectively. Cells corresponding to prolines (for which no back-bone amide protons are present) appear in gray.
^{c}Assignments are tentative

### Mapping of Binding Interface Using the Cross-Saturation Method

For distinct mapping of the contact surface between PRL-G129R and ECD-PRL-R the cross-saturation method was applied. Thus, a pair of 2D-1H, ¹⁵N-TROSY spectra of the complex between [²H, ¹⁵N]PRL-G129R and ECD-PRL-R was recorded, one spectrum with saturation applied in the aliphatic region (at 0.9 ppm) and one reference spectrum with the saturation field applied well off-resonance (−5 ppm). Cross-saturation experiments were recorded with different lengths of the saturation period (0.5 and 1 second) and with different contents of ²H₂O in the buffer (90 and 95%).

Peak intensities in the two spectra were measured and the ratio of the peak intensity in the spectrum with saturation relative to the intensity of the corresponding peak in the reference spectrum was calculated. Signals displaying strong attenuation in the saturated spectrum are attributable to the residues in PRL-G129R for which the corresponding amide proton is in close proximity (<7 Å distance) of protons in the receptor chain, and therefore are likely to be important for receptor interaction.

Strongest attenuation (>40%) of the amide signal in the cross-saturation experiment was observed for the following residues

S26, S33, I55, N56, D68, K69, M130, K142, R177, D178, K181, I182, D183, L189, I194 and C199.

Strong attenuation (25-40%) was also observed for several other residues including: I51, E67, A72, S90, L98, V99, Q106, V117, L126, L127, L132, T141, Y169, S179, H180, L186 and L188.

The complete data from the cross-saturation experiment are shown in FIG. 9, where the observed signal intensity ratio of the amide proton signal for each amino acid residue is plotted against residue number. Residues exhibiting strongest attenuation (>40%) are mapped on the 3D PRL structure (pdb-code 1RW5) in FIG. 10.

### Description of the Binding Interface Between PRL-G129R and ECD-PRL-R

A large number of residues distributed throughout the primary sequence was recorded as perturbed in the chemical shift perturbation experiment. These residues are affected either by direct contacts with the receptor at the binding interface, or by secondary effects (conformational changes) induced by binding. The large number of perturbations, including several buried residues indicates that structural rearrangements of PRL are induced by receptor binding. However, the magnitude of the structural perturbations can not be deduced from the present data, and might only be subtle.

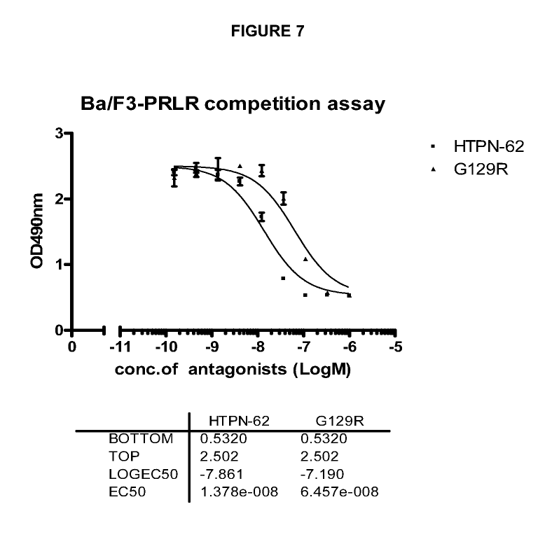

Teilum (2005) observed that NMR signals for amide groups were absent in stretches in several regions of the protein due to flexibility and fast exchange of the amide protons. Thus, save for I3, no amide resonances were observed for residues 1-15 in free PRL, which is also the case for free PRL-G129R and for the complex between PRL-G129R and ECD-PRL-R. This indicates that the flexible N-terminal part is not involved in or influenced by binding. Included in the NMR silent regions in PRL are also a part of the loop between helix 1 and helix 2 (H46-H59), a part of the loop between helix 3 and helix 4 (S151-A159), and the C-terminal segment (H195-N198). As shown in FIG. 7 (green bars) several residues situated in these regions (I51-S57 and H195-N198) display amide proton resonances in the complex between PRL-G129R and ECD-PRL-R, indicating that that they become shielded, stabilized, or by some other means, protected from solvent exchange upon complex formation. Thus, these observations point particularly to two regions, I51-S57 and H195-C199, as being important for receptor interaction.

### Example 3

Two hotspot libraries were generated with Error-prone PCR using PRL G129R as the template. The libraries were screened with Scintillation Proximity Assay (SPA). About 1% of the hits were cherry picked and confirmed with SPA. About 10% of the hits identified by confirmation SPA were purified and analyzed with Biacore assay and cell-based bioassay. Two hits, [PRL Q73L, M75T, N76S, F80L, G129R] and [PRL S33A, Q73L, G129R, K190R], were identified to have higher affinity than wt PRL and 6 to 8 fold higher antagonist activity compared with PRL G129R. Also the following sites were discovered to have positive effect on the affinity of PRL to PRLR: L25Q, Y28N, N31S, S33A, D68N, Q73L, M75T, N76S, F80L, and S179T, K190R.

### Library Generation:

The library LibMixNew was generated based on EZclone strategy (Genemorphll EZclone Domain Mutagenesis Kit, stratagene catalog # 200552). Mixture of primer Lib23-83 and Lib173-199 which generated by error prone PCR were used as mega primer for round the world PCR by pfu polymerase. After Dpnl digestion, 6 separate reactions were performed as 3 μl of PCR product were transformed into 50 μl DH5 competent cell, recover 20 mins at 37° C., Plate on LBA plate for 0/N at room temperature. Collect around 50,000 colonies from all the plates for plamid purification, 10 μg plamid can be recovered. 100 ng were transformed into host strain Origami, recover at 20 min at 37° C., plate on LBA plate at 37° C. for overnight to get the clones for screening. According to sequence analysis result based on Origami cell, mutation efficiency of the library is 86%.

### Culture Process

Seeds were prepared by inoculate clones from plate by QPix2 colony picker into 96-deep well plate, culture at 37° C. for overnight, by this way cell density of seeds can be normalized to saturated stage of OD600 2.0. Transfer 20 μl seed from overnight culture deep well plate into 230 μl of LBA broth into the 96-deep well plates by liquid handler, Incubate all plates in Shaker flask at 37° C. with shaking at 220 rpm for 2.5 hrs to OD600=0.8, add 10 μl of 500 μM IPTG stock to each well with Fill Liquid Dispenser, incubate all 24 96-deep well plates in Shaker at 25° C. with shaking at 220 rpm for overnight (16 hrs).

### Preparation of BirA-Ser-PRLR (1-210)

The pET39b-BirATag-Ser-PRLR(1-210)/E. coli BL21(DE3) was cultivated at 37° C. in LB medium supplemented with 25 μg/ml of Kanamycin and 10 μg/ml of chloramphenicol to an optical density of 0.8, and the cells were induced with 0.5 mM IPTG and 100 μM biotin for 6 hours (37° C., 250 rpm). The cell pellet was harvested by centrifugation, resuspended in the buffer (20 mM Tris, pH 8.0, 5 mM EDTA, 2 mM DTT, 0.05% Tween 20) and disrupted with the cell disruptor (Z-plus, Constant Systems). The inclusion bodies were pelleted and solubilised with 100 mM Tris, pH 8.0, 8 M urea, 5 mM DTT. The solubilised material was clarified by centrifugation, then diluted 20-fold into the refolding buffer (20 mM Tris, pH 8.0, 0.05% Tween 20, 0.5 mM GSSH, 0.1 mM GSSG) and stirred at 16° C. for 65 hours. The refolded protein was purified with QHP sepharose (GE), followed by affinity purification with SoftLink™ Soft Release Avidin Resin (Promega).

### SPA Assay:

The cells in 96-well plates were harvested by centrifugation. The cell pellet was resuspended with the lysis buffer (CelLytic Express, Sigma) and stayed at RT for 1 hr for complete lysis. The cell lysate was diluted with pure water 3 times. 15 μl of the lysate was added into 85 μl of the assay buffer (50 mM Tris, pH 8.0, 0.05% Triton X-100, 0.2% BSA) containing 0.3 mg streptavidin SPA beads (RPNQ0066V, GE), 0.1 μCi of tritium labelled wt PRL and 150 nM BirA-Ser-PRLR (1-210). Stay at room temperature for 3 hours and count with the luminescence counter (MicroBeta TriLux, PerkinElmer). The pipetting was performed with the liquid handler (Biomek FX, Beckman).

### Purification of the Hits:

The pET32_PRL mutant (hits)/E. coli Origami was cultivated at 37° C. in LB medium supplemented with 100 μg/ml Ampicillin to an optical density of 0.8˜1.0, and the cells were induced with 50 μM IPTG overnight. The cell pellet was harvested by centrifugation, and then lysed with the lysis buffer (CelLytic Express, Sigma). The cell lysate was clarified by centrifugation and purified with Ser-PRLR (1-210) coupled sepharose 4 FF (NHS-activated sepharose 4 FF, GE).

### Biacore Assay:

Biotinylated prolactin receptor BirATag-Ser-PRLR (1-210) was diluted to 20 μg/ml in 10 mM sodium acetate pH 4.0 (Biacore BR-1003-49) and immobilized on the CM5 chip (Biacore BR-1006-68) with the immobilization reagents 1-ethyl-3-(3-dimethylaminopropyl) carbodiimide hydrochloride (EDC), N-hydroxysuccinimide (NHS) and 1.0 M ethanolamine-HCl pH 8.5 (Biacore BR1000-50). The immobilization level was 1500 RU. The PRL analogues were diluted to a series of concentrations as following: 1.6/3.13/6.25/12.5/25 nM and run through the PRLR immobilized chip using HBS-EP (10 mM HEPES pH 7.4; 150 mM NaCl; 3 mM EDTA; 0.005% v/v Tween-20) as the running buffer under the following conditions:

Sample: contact time 300 s; flow rate 40 ul/min; dissociation time 180 s.

Regeneration: contact time 50 s; flow rate 20 ul/min; stabilization period: 5 s

4.5 M MgCl₂ was used as the regeneration buffer.

### PRLR Stably Expressing Ba/F3 Cell Line Generation (Ba/F3-PRLR)

To generate a cell based reference bio-assay for PRLR antagonist evaluation, Ba/F3 cells were transfected with PRLR gene containing plasmid. Cells could survived under 1 ng/ml wtPRL stimulation were subcloned and 48 clones with fast growing were chosen for further dose-response study under different wtPRL stimulation. About 50% of these 48 clones can survive at 0.4 ng/ml wtPRL but only 2 of them kept proliferation at 0.1 ng/ml wtPRL. In the stimulation assay, these two cell lines (PRLR-09 and PRLR-32) showed good response to wtPRL (PRLR-09 EC50: 5.4E-11 M and PRLR-32 EC50: 3.75 E-11 M). And both the cell lines could reach the maximal biological response under 1 nM wtPRL (23 ng/ml) stimulation, which is very similar to the reported data (10 ng/ml) of Ba/F3-PRLR cell model successfully used for the same aim.

### Agonist/Antagonist Bioassays on PRLR Ba/F3 Cell Line:

The Ba/F3-PRLR cells are grown on starvation medium (RPM I 1640 with 10% FCS) for 24 hours. The cells then were resuspended in starvation medium to 5×10⁵ cells/ml, 100 μl of the cells are feed into 96-well plate, 50 μl of agonist or wtPRL(1 nM)/antagonists in different conc. are added into the cells, and incubated for 68 hours. 50 μl of AlamarBlue (starvation medium: AlamarBlue reagent=7:1) is added to each well, and then incubated for 4 hours. The Fluorescence was measured with the plate BMG LABTECHNOLOGIES 96, the excitation filter of 544 nm and the emission filter of 590 nm. Prism4 software was used to analyze the data.

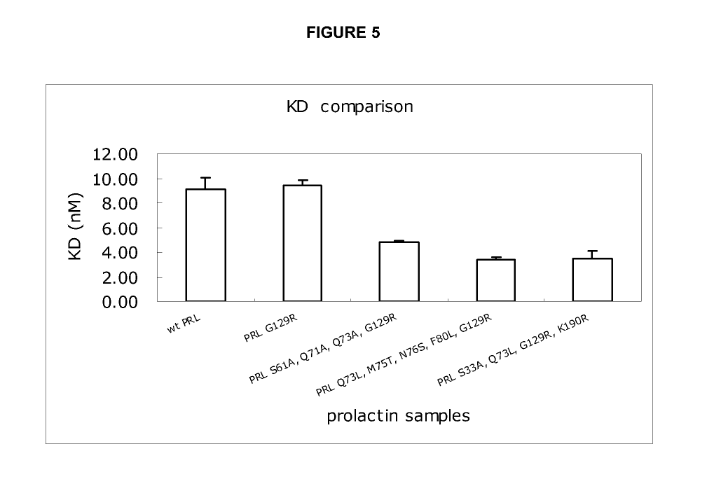

FIG. 5 shows the Biacore assay results of some prolactin analogs. [PRL S61A, Q71A, Q73A, G129R] was a rational designed mutant. [PRL Q73L, M75T, N76S, F80L, G129R] and [PRL S33A, Q73L, G129R, K190R] were two hits identified by SPA assay. The result indicated that the affinity of the two hits was almost 2-fold higher than wt PRL and [PRL G129R].

FIG. 6 shows a Ba/F3-PRLR proliferation assay result. The wtPRL reached highest stimulation activity around 1 nM and the EC50 is 1.02E-10M. The PRL-G129R reached highest stimulation activity around 110 nM and the EC50 is 3.2E-09M. The highest proliferation rate under PRL-G129R stimulation is only 12% that of wtPRL. For the rabbit poly-clonal Ab anti-hPRLR and two PRL mutants, very weak agonist activity could be detected.

FIG. 7 shows an example of Ba/F3-PRLR competition assay result. HTPN-62 is the mutant [PRL Q73L, M75T, N76S, F80L, G129R], one of the hits identified by SPA assay and Biacore assay. The result indicated that the antagonist activity of the mutant was about 4 fold higher than that of [PRL G129R].

### Example 4

### KD of Selected Prolactin Variants

Biotinylated prolactin receptor BirATag-Ser-PRLR (1-210) was diluted to 20 μg/ml in 10 mM sodium acetate pH 4.0 (Biacore BR-1003-49) and immobilized on a CM5 chip (Biacore BR-1006-68) with the immobilization reagents 1-ethyl-3-(3-dimethylaminopropyl) carbodiimide hydrochloride (EDC), N-hydroxysuccinimide (NHS) and 1.0 M ethanolamine-HCl pH 8.5 (Biacore BR1000-50). The immobilization level was 1500 RU. The PRL variants prepared as described in Example 5 were diluted to a series of concentrations as following: 3.13/6.25/12.5/25/50 nM and run through the PRLR immobilized chip using HBS-EP (10 mM HEPES pH 7.4; 150 mM NaCl; 3 mM EDTA; 0.005% v/v Tween-20) as the running buffer under the following conditions:

Sample: contact time 240 s; flow rate 40 ul/min; dissociation time 180 s.

Regeneration: contact time 50 s; flow rate 20 ul/min; stabilization period: 5 s

4.5 M MgCl₂ was used as the regeneration buffer.

The KD of selected variants of human prolactin can be seen in Table 2.

TABLE 2

| | | KD | |
|---|---|---|---|
| No. | PRL variant | (nM) | STDEV |
| 1 | Ser-PRL Q73L M75T N76S F80L G129R | 3.6 | 0.28 |
| 2 | Ser-PRL S33A Q73L G129R K190R | 5.0 | 0.064 |
| 3 | Ser-PRL G129R, K190R | 5.1 | 0.11 |
| 4 | Ser-PRL S61A, G129R, K190R | 2.4 | 0.028 |
| 5 | Ser-PRL Q73L, G129R, K190R | 2.6 | 0.17 |
| 6 | Ser-PRL S33A, Q73L, M75T, N76S, F80L, G129R, K190R | 3.3 | 0.51 |
| 7 | Ser-PRL S33D, Q73L, M75T, N76S, F80L, G129R, K190R | 3.1 | 0.17 |
| 8 | Ser-PRL D68N, G129R | 4.9 | 0.56 |
| 9 | Ser-PRL D68N, G129R, K190R | 2.8 | 0.22 |
| 10 | Ser-PRL S38I, D68N, G129R, S179T, I182V | 3.1 | 0.29 |
| 11 | Ser-PRL G129R, K190R, I194V, H195Y, N196R, N197D | 3.2 | 0.042 |
| 12 | Ser-PRL K42R, Q73L, G129R, K190R | 2.6 | 0.092 |
| 13 | Ser-PRL S33A, Q73L, G129R, K190R, I194V, H195Y, N196R, | 3.8 | 0.44 |
| | N197D | | |
| 16 | PRL S61A, D68N, G129R, K190R | 1.4 | 0.000 |
| 17 | Ser-PRL D68N, Q73L, G129R, K190R | 1.2 | 0.057 |
| 18 | Ser-PRL D68N, E70K, Q73L, G129R, K190R | 1.2 | 0.021 |
| 19 | Ser-PRL S61A, D68N, Q73L, G129R, K190R | 1.1 | 0.085 |
| 20 | Ser-PRL S61A, Q73L, G129R, K190R | 1.3 | 0.049 |
| 21 | PRL S61A, D68N, G71A, Q73A, G129R, K190R | 1.7 | 0.035 |
| 22 | Ser-PRL S38I, D68N, G129R, I182V | 2.8 | 0.18 |
| 23 | Ser-PRL S61A, D68N, G129R, K190R | 2.3 | 0.1273 |
| 24 | Ser-PRL S38I, S61A, D68N, Q73L, G129R, K190R | 0.3 | 0.0636 |
| 25 | Ser-PRL N31R, G129R, K190R | 2.01 | 0.3606 |
| 26 | Ser-PRL N31E, G129R, K190R | 2.70 | 0.2546 |
| Control | PRL G129R/Ser-PRL G129R | 13.9 | 1.63 |

### Example 5

### Protein Expression and Purification

The pET32-a(+) expression vector (Novagen, Madison Wis.) was used for expression of proteins. Recombinant ECD, PRL and mutated PRL monomers were produced as inclusion bodies in Escherichia coli BL21(DE3) cells co-transfected with pACYCDuet-MetAP plasmid, which express the E. coli MetAP protein. Solubilized in 8M urea, 0.1 M Tris, 2-20 mM DTT, pH 8.5 buffer and following refolding by dilution into a 20 mM Tris, 0.05% Tween 20, pH 8.0. Protein purification was performed using Source30Q ion exchange columns (Amersham Biosciences) followed by a macro-prep Caramic Hydroxyapatite column (BioRad) and a final size-exclusion chromatography on a Sephadex G25 column. PRL receptor was refolded in two dilution steps, first in 0.4M arginine pH 8.5 and then diluted further in 20 mM Tris, 0.05% Tween 20, pH 8.0.

### Pharmacological Methods

### Assay (I)

### Prolactin Receptor Binding Assessed by Surface Plasmon Resonance Measurements

Test compound, in this case ECD-PRL-R (25 μg/ml in 10 mM sodium acetate, pH 3.0), was injected into a Biacore 3000 instrument at a flow rate of 5 μl/min and coupled to a CM5 sensor chip by amine coupling chemistry. Prolactin and variants thereof (500 nM in buffer; 20 mM Hepes, pH 7.4, containing 0.1 M NaCl, 2 mM CaCl₂ and 0.005% P20) were then injected over the immobilized receptor for 5 minutes at the same flow rate, followed by a 10-min dissociation period during which buffer was injected, to assess receptor binding affinity. Data evaluation was performed in BiaEvaluation 4.1. Regeneration was accomplished with 4.5 M MgCl₂ between runs.

### Assay (II)

### Determining Antagonism/Agonism for the PRL Receptor Using a STAT5 Reporter Assay.

AU 565 cells were cultured for 2 days in 6-well dishes. Cells were starved for 18 hours in growth medium with <1% FCS prior to treatment with PRLR binding compounds. The cells were incubated for 15 min at 37° C. in a humidified CO₂ incubator after addition of compounds. Cell lysate was prepared and analyzed for STAT5 tyrosine phosphorylation by Western blotting using an anti-STAT5 [pY694] specific antibody (Cell Signalling Technologies).

### Assay (III)

### Phospho-STAT3 ELISA

T47D cells grown to approximately 80% confluency were detached with trypsin; cell density was adjusted to 5×10⁵/ml in full growth medium (RPMI, 10% FCS, 2 mM L-glutamin, 0.2 U/ml bovine insulin). 200 μl of this suspension was plated per well of a 96-well plate. The next day, growth medium was replaced with 150 μl starvation medium (growth medium omitting 10% FCS). The cells were starved for 24 hours prior to treatment with PRLR binding compounds. PRL and inhibitors were pre-mixed in starvation medium and 50 μl were added per well to result in 10 nM PRL and varying concentrations of inhibitors indicated at FIG. 8. The cells were incubated for 15 min at 37° C. in a humidified CO₂ incubator. Medium was removed and the cells were washed with ice-cold PBS. Lysis of cells and ELISA were performed according to BioSource STAT-3 [pY705] phospho ELISA manual.

### Assay (IV)

### Agonist/Antagonist Bioassays on PRLR Ba/F3 Cell Line:

The Ba/F3-PRLR cells are grown on starvation medium (RPM I 1640 with 10% FCS) for 24 hours. The cells then were resuspended in starvation medium to 5×10⁵ cells/ml, 100 μl of the cells are feed into 96-well plate, 50 μl of agonist or wtPRL(1 nM)/antagonsits in different conc. are added into the cells, and incubated for 68 hours. 50 μl of AlamarBlue (starvation medium: AlamarBlue reagent=7:1) is added to each well, and then incubated for 4 hours. The Fluorescence was measured with the plate BMG LABTECHNOLOGIES 96, the excitation filter of 544 nm and the emission filter of 590 nm. Prism4 software was used to analyze the data.

1. An isolated peptide, which peptide is a variant of a PRL-like cytokine, said variant comprising
(i) one or more amino acid mutations in the region corresponding to amino acid residue 24 to 35 of SEQ ID No. 1 and/or
(ia) one or more amino acid mutations in the region corresponding to amino acid residue 52 to 58 of SEQ ID No. 1 and/or
(ib) one or more amino acid mutations in the region corresponding to amino acid residue 50 to 57 of SEQ ID No. 1 and/or
(ii) one or more amino acid mutations in the region corresponding to amino acid residue 66 to 83 of SEQ ID No. 1 and/or
(iii) one or more amino acid mutations in the region corresponding to amino acid residue 176 to 199 of SEQ ID No. 1 and/or
(iv) an addition of from 1 to 5 amino acid residues to the C-terminal.

2. An isolated peptide according to claim 1, wherein at least one of the mutation(s) described under (ia) is in the position corresponding to amino acid residue 51 of SEQ ID No. 1.

3. An isolated peptide according to claim 1, wherein at least one of the mutation(s) described under (ia) is in the position corresponding to amino acid residue 55 of SEQ ID No. 1.

4. An isolated peptide according to claim 1, wherein at least one of the mutation(s) described under (ia) is in the position corresponding to amino acid residue 56 of SEQ ID No. 1.

5. An isolated peptide according to claim 1, wherein at least one of the mutation(s) described under (ia) is in the position corresponding to amino acid residue 57 of SEQ ID No. 1.

6. An isolated peptide according to claim 1, wherein the PRL-like cytokine has at least 80%, such as at least 85%, for instance 90%, such as 95%, for instance 96%, such as 97%, for instance 98%, such as 99% identity to the amino acid sequence of human prolactin, growth hormone, placenta lactogen, interleukin-2, interleukin-3, interleukin-4, interleukin-6, interleukin-17, interleukin-20, interleukin-21, interleukin-31, interleukin-32 or erythropoietin (EPO).

7. An isolated peptide according to claim 1, wherein the PRL-like cytokine has at least 80% identity to SEQ ID No. 1.

8. An isolated peptide according to claim 7, wherein the PRL-like cytokine comprises the amino acid sequence of SEQ ID No. 1.

9. An isolated peptide according to claim 1, wherein the PRL-like cytokine has at least 80% identity to SEQ ID No. 2.

10. An isolated peptide according to claim 9, wherein the PRL-like cytokine comprises the amino acid sequence of SEQ ID No. 2.

11. An isolated peptide according to claim 1, wherein the PRL-like cytokine has at least 80% identity to SEQ ID No. 3.

12. An isolated peptide according to claim 11, wherein the PRL-like cytokine comprises the amino acid sequence of SEQ ID No. 3.

13. An isolated peptide according to claim 1, wherein at least one of the mutation(s) described under (i) is in the position corresponding to amino acid residue 25, 28, 31, 33, 68, 70, 75, 76, 80, 182, 190, 194, 196, 197 of SEQ ID No. 1.

14. An isolated peptide, which peptide is a variant of a PRL-like cytokine, said variant comprising one or more amino acid mutations, which stabilizes the structure of the prolactin molecule.

15. An isolated peptide according to claim 14, wherein the PRL-like cytokine has at least 80% identity to SEQ ID No. 1.

16. An isolated peptide according to claim 15, wherein the PRL-like cytokine comprises the amino acid sequence of SEQ ID No. 1.

17. An isolated peptide according to claim 14, wherein the PRL-like cytokine has at least 80% identity to SEQ ID No. 2.

18. An isolated peptide according to claim 17, wherein the PRL-like cytokine comprises the amino acid sequence of SEQ ID No. 2.

19. An isolated peptide according to claim 14, wherein the PRL-like cytokine has at least 80% identity to SEQ ID No. 3.

20. An isolated peptide according to claim 19, wherein the PRL-like cytokine comprises the amino acid sequence of SEQ ID No. 3.

21. An isolated peptide according to claim 1, wherein said peptide comprises one or more amino acid mutations, which stabilizes the secondary structure of the prolactin molecule.

22. An isolated peptide according to claim 1, wherein one or more of said amino acid mutations are selected from mutations in the amino acid residues corresponding to Ala-111 and Glu-162.

23. An isolated peptide according to claim 1, wherein one or more of said amino acid mutation(s) introduces salt bridges in helical segments exposed to solvent.

24. An isolated peptide according to claim 1, wherein one of said amino acid mutations is a mutation in the amino acid residue corresponding to Asn-92.

25. An isolated peptide according to claim 1, wherein two or more of said amino acid mutation(s) introduces non-native disulfide bonds into prolactin.

26. An isolated peptide according to claim 1, wherein one or more of said amino acid mutation(s) is a substitution of a solvent exposed hydrophobic residue with a polar residue.

27. An isolated peptide according to claim 26, wherein one or more of said amino acid mutations are selected from mutations in the amino acid residues corresponding to Ile-146 and Val-149.

28. An isolated peptide according to claim 1, wherein one or more of said amino acid mutation(s) improves the packing interactions at the hydrophobic core of the 4-helix bundle structure.

29. An isolated peptide according to claim 28, wherein one or more of said amino acid mutations are selected from mutations in the amino acid residues corresponding to Leu-95, Ile-119 and Leu-175.

30. An isolated peptide according to claim 1, wherein said peptide has an increased affinity to the prolactin receptor as compared to human prolactin.

31. An isolated peptide according to claim 1, wherein said peptide is capable of binding to the human growth hormone receptor.

32. An isolated peptide according to claim 1 also comprising at least one amino acid substitution selected from an amino acid mutation in the position corresponding to position 61, an amino acid mutation in the position corresponding to position 71 and an amino acid mutation in the position corresponding to position 73 of SEQ ID No. 1.

33. An isolated peptide according to claim 1, which peptide have been modified so that binding of the peptide via BS2 to the prolactin receptor is disrupted.

34. An isolated peptide according to claim 33, wherein at least one of said disruptive mutations is a mutation in the amino acid residue corresponding to Gly-129 in SEQ ID No. 1.

35. An isolated peptide according to claim 34, wherein the amino acid residue corresponding to Gly-129 in SEQ ID No. 1 has been substituted with an Arg.

36. An isolated peptide according to claim 1, wherein the amino acid residues corresponding to positions 1 to 9 in PRL have been deleted.

37. An isolated nucleic acid encoding a peptide according to claim 1.

38. A vector comprising a nucleic acid construct according to claim 37.

39. A host cell comprising a a vector of claim 38.

40. An antibody that specifically binds a peptide according to claim 1.

41. An antibody according to claim 40, which antibody does not bind to a peptide comprising the amino acid sequence of SEQ ID No. 1, SEQ ID No. 2 or SEQ ID No. 3.

42. A pharmaceutical formulation comprising a peptide according to claim 1.

43. A peptide according to claim 1 for use in therapy.

44. A peptide according to claim 43 for use in treating or preventing a proliferative disorder.

45. A peptide according to claim 44, wherein said proliferative disorder is a cancer.

46. A pharmaceutical formulation comprising a peptide according to claim 1.

47. A pharmaceutical formulation according to claim 46 for use in the treatment or prevention of a proliferative disorder.

48. (canceled)

49. (canceled)

50. (canceled)

51. A method of treatment or prevention of a proliferative disorder, which comprises administration of an effective amount of a peptide according to claim 1 to a patient in need thereof.
